(19) United States
(12) Patent Application Publication
(10) Pub. No.: US 2014/0093942 A1
(43) Pub. Date: Apr. 3, 2014

(54) GENE, ARS-R ANCHORAGE CASSETTE, ARS-R EXPRESSION-ANCHORAGE CASSETTE, RECOMBINANT PLASMID, BACTERIAL TRANSGENIC LINEAGE, USE OF SAID GENE, USE OF SAID LINEAGE IN ENVIRONMENTAL BIOREMEDIATION PROCESSES

(71) Applicants: UNIVERSIDADE DE SÃO PAULO - USP, Sao Paulo (BR); VALE S.A., Rio de Janeiro (BR)

(72) Inventors: Elisabete José VICENTE, Jandira (BR); Ana Clara Guerrini Schenberg, Sao Paulo (BR); Carolina Angélica da Silva Parada, Sao Paulo (BR); Ronaldo Biondo, Sao Caetano do Sul (BR)

(21) Appl. No.: 13/902,477

(22) Filed: May 24, 2013

Related U.S. Application Data
(60) Provisional application No. 61/651,906, filed on May 25, 2012.

(51) Int. Cl. C07K 14/195; B09C 1/10

(52) U.S. Cl. CPC C07K 14/195; B09C 1/10
USPC 435/252.33; 536/23.7; 435/320.1; 435/252.3; 435/262.5

(57) ABSTRACT

The present invention relates to the construction and insertion of a DNA plasmid vector of broad spectrum for Gram-Negative bacteria, that carries a gene sequence which, when expressed, enables the anchorage of a chelator protein for arsenic ions on the Gram-Negative bacteria cellular surface. For that end, the structural sequence of the regulatory arsR gene without stop codon (SEQ ID No 1) was amplified by Polymerase Chain Reaction (PCR) using as a template the chromosome 1 of Cupriavidus metallidurans, CH34 lineage and inserted into the pGEM-T cloning vector, yielding the pGEMT-As plasmid (SEQ ID No 2). The expression vector containing the sequence encoding the cassette for the expression and anchoring of heterologous proteins in Gram-negative bacteria, under the control of the pan promoter (SEQ. ID No 3) was obtained upon digestion of the pCM-Hg plasmid with XbaI and SalI restriction enzymes. The arsR gene was released from the pGEMT-As plasmid by digestion with XbaI and SalI restriction enzymes and then ligated to the linearized expression vector, called pCM (SEQ. ID No 4), resulting in the construction of the pCM-As plasmid (SEQ ID No 5). Additionally, the present invention provides recombinant strains of Gram-negative bacteria containing said recombinant plasmid, method of production, use of the recombinant plasmid to enhance bacterial arsenic resistance and capability to adsorb arsenic ions, as well as the use of the transgenic strains for the adsorption of arsenic ions in environmental bioremediation processes, with the possibility of recovering the metalloid as a byproduct.

### FIELD OF INVENTION

The present invention relates to the construction and insertion of a broad spectrum vector for Gram-negative bacteria carrying a gene sequence which, when expressed, allows the anchorage of a chelating protein of arsenic ions on the cellular surface of Gram-negative bacteria. Additionally, the present application provides recombinant strains of Gram-negative bacteria containing said recombinant plasmid, a method for obtaining them, besides reporting the potential use of the recombinant strains for arsenic ions adsorption in environmental bioremediation processes.

### BACKGROUND OF THE INVENTION

Arsenic (As) is a metalloid with oxidation states of 3⁻, 0, 3⁺ and 5⁺. This element is found in low concentrations in nature, in rocks, volcanic regions, in sediment and marine fauna and flora. It occurs especially in the organic and inorganic forms, as a result of its participation in biological and chemical complex processes. Among the volatile forms, arsine is found in the atmosphere (AsH₃), while the elementary arsenic (As⁰) is of rare natural occurrence (Soluble species of arsenic are found in the hydrosphere. In natural waters, the arsenic can occur as arsenite (As³⁺) arsenate (As⁵⁺), monometilarsonic ion (MMA) and dimethylarsinic ion (DMA). Groundwaters contain As³⁺ and As⁵⁺.

In sea waters, ponds, lakes and where there is a possibility of biomethylation, As³⁺ and As⁵⁺ occur along with MMA and DMA. The marine flora and fauna contain arsenic compounds, since in the metabolic routes, nitrogen and phosphorus can be easily replaced by it. Such compounds also include, besides the arsenobetaine, arsenocoline and arsenosugars of algal source. In mineral deposits, the metalloid is found mainly as arsenopyrite (FeAsS) and arseniferous pyrite which may alter to arsenates and sulfo-arsenate in the surface, the arsenic can be partially released into the water and still be immobilized via adsorption in iron oxides-hydroxides, aluminum and manganese or clay minerals.

Most forms are toxic. The decreasing order of arsenic compounds toxicity is as follows: arsine>arsenite>arsenate>alkyl arsenic acids>arsonium compounds>elementary arsenic. The inorganic compounds are 100 times more toxic than the partially methylated forms (MMA and DMA). Arsenobetaine and arsenocoline are relatively non-toxic.

However, high concentrations of arsenic in the environment are the result of various anthropogenic activities such as: combustion of fossil fuels, application of pesticides, fungicides, fertilizers and wood preservatives, glass, cement and semiconductors manufacturing, it is also emitted as a byproduct of copper, zinc and lead refining, gold mining industries dumping of industrial effluents and improper disposal of “e-waste” such as televisions, cell phones, batteries, and computer components.

After the death of Napoleon Bonaparte by arsenic poisoning in 1821, the first cases of severe mass poisoning were reported in Bangladesh and West Bengal (India), due to the exposure of approximately 58 million people through the consumption of contaminated water extracted from aquifers in arsenical geological formations of large extensions. Similar cases have been reported in Chile, Argentina, Mexico, Spain and Taiwan.

The increasing industrial activity in China has led to intensive combustion of mineral coal in the Southwest of the country that resulted in high levels of arsenic release in the atmosphere with the consequent poisoning of the local population.

In the United States of America, regions with artesian wells industrially impacted have been reported in Michigan and Wisconsin, as well as in water recreation areas in the north of Boston. It is estimated that 20 million North Americans are consuming contaminated water with arsenic compounds. According to the “Agency for Toxic Substances and Disease Registry” (ATSDR), the metalloid is found in the top of the list of the most dangerous substances.

In Brazil, the natural sources contaminated by arsenic are related to the rocks that host sulphidic gold deposits, such as the Iron Quadrangle (Quadriláter® Ferrifero) region (MG), the Fazenda Brasileiro (Teofolândia-BA), the Mina III (Crixás—GO) and the Vale do Ribeira (SP). The anthropogenic sources already identified in Brazil are localized and are related to ore mining and refining activities of some of the gold deposits mentioned above. The Quadrangle Iron has alone been responsible for the production of 1,300 tons of gold (Au⁺) in the last three centuries, and considering the ratio As/Au in the ores, it is estimated that at least 390,000 tons of As must have been released into the environment.

Arsenic is an extremely toxic metalloid, being the inorganic forms (As³⁺ and As⁵⁺) the most harmful to humans for its genotoxicity and consequent carcinogenicity. In vivo, it reacts with thiol groups of proteins and produces oxidative species that cause severe cellular damages and chromosomal aberrations. Furthermore, the inorganic forms have the ability to cross barriers in the membranes of living beings, causing drastic effects in low concentrations, such as cardiovascular diseases and neurological disorders, severe encephalopathy, hemolysis, bone marrow depression, spontaneous miscarriages, mellitus diabetes, various neoplasms types, numerous of other serious illnesses and even death from poisoning.

According to the values established by the World Health Organization (WHO), the total metalloid concentration should not exceed 0.02 to 4 ng/m³ in the air, 1 to 2 μg/L in ocean waters, 10 Ξg/L in rivers and ponds, with the exception of volcanic regions and natural sulfide deposits that can have higher limits. Likewise, high levels of arsenic can be found in the ground (1-40 mg/kg) due to the geological composition and the presence of sulphides. Contaminated soils by anthropogenic activities can reach contamination levels in the order of 100 mg/Kg.

In Brazil, the resolution of the National Environment Council (CONAMA), CONAMA 357/2005, establishes that the total arsenic value should not exceed 0.01 mg/L in class 1 fresh, saline and brackish waters, 0.069 mg/L in class 2 saline and brackish waters, and 0.033 mg/L in class 3 fresh water. In relation to the disposal of effluents, the resolution establishes a maximum total arsenic value of 0.5 mg/L.

Law No. 9605, of Feb. 12, 1998, provides for criminal and administrative sanctions to conduct and activities that are harmful to the environment. Nonetheless, many Brazilian waterways have a high mutagenic potential due to the presence of toxic contaminants such as heavy metals, that are inadvertently discarded.

In addition, in order to mitigate “e-waste” environmental contamination, Law No. 12.305/10 and Resolution No. 401/08 were regulated. The Senate Bill 714/2007 has been recently approved, which provides for the final collection and destination of used batteries.

In the United States, the “Environmental Protection Agency” (EPA) sets out the safe concentrations of up to 10 parts per billion (ppb) in water available for human consumption, besides focusing on the development and evaluation of innovative and economically feasible methods for controlling contamination. The “Food and Drug Administration” (FDA) establishes the maximum values of inorganic arsenic in food, with special attention to crustaceans, among other seafood due to the presence of metalloid in marine sediments.

In the European Union, the concern about contamination levels combined with scarcity of water resources has forced an improvement of the environmental legislation, limiting the disposal of wastewater toxic contaminants, including heavy metals, forcing the various productive sectors to implement advanced treatment technologies.

Despite the established limits and the current environmental laws and regulations, since ancient times, the amount of information in the literature describing the diversity of contaminated sites with arsenic compounds as a result of anthropogenic activities and improper disposal of products and effluents has steadily increased worldwide, turning it not only into an environmental problem, but also a public health issue.

The decontamination of polluted sites is one of the biggest challenges to sustainable development. Among the methods that can be used to remediate arsenic contaminated environments are the available physicochemical techniques, which involve precipitation processes, ionic exchange, adsorption and solvent extraction. Subsequent processes such as sedimentation and filtration are generally required for the treated water to be recovered. However, besides being economically unviable, they destroy the natural landscape, result in sludge with high content of heavy metal with no set destination, and can affect the health of people directly involved in the process

The search for remediation processes which are economically viable and environmentally friendly have been intensified in recent years, bioremediation has been described as an attractive alternative. When compared to conventional processes, bioremediation presented the following advantages: a) the biosorbents can be produced with low cost, b) they are reusable, c) they can provide high amounts of metal accumulation d) they may present selectivity to specific metals, and, e) when immobilized, the separation of the solution is efficient and fast.

Bioremediation is the process by which living organisms, whether viable or not, modified or not, are used to remove or reduce pollutants in the environment, said living organisms being organic or heavy metals.

The prolonged exposure of some bacterial strains to arsenic contaminated sites has led certain communities present in these areas to improve their resistance in order to survive by developing specific cellular detoxification mechanisms. Numerous studies have been conducted aiming to understand the functioning of such naturally developed biological systems and to prospect new potentially resistant strains.

A considerable variety of bacteria with distinct degrees of resistance and capable of adsorbing heavy metals have been described.

This multiplicity of lineages and resistance mechanisms is enabling the use of these microbes in bioremediation strategies, either in-situ (at the contaminated area), or ex-situ (involving the removal of contaminated material to be treated somewhere else). Some bacteria have already been employed in biological processes and have proved effective in the recovery of contaminated areas.

Arsenic resistant bacteria have developed different strategies for arsenic biotransformation, including arsenite oxidation (As³⁺), cytoplasmic arsenate reduction (As⁵⁺), respiratory reduction of As⁵⁺ and As³⁺ methylation. The primary function of these transformations is to ensure cell survival in sites containing high concentrations of this toxic metalloid. Therefore, plasmids containing genes that confer resistance have been isolated from the bacteria. Arsenic resistance determinants, called ars genes, can be found in Gram-positive and Gram-negative bacteria, consisting of genes arranged in a single transcriptional unit, called ars operon.

The Gram-negative bacterium Acidithiobacillus ferrooxidans has proved efficient for the removal of arsenic organic forms. However, there is a need for decontamination of inorganic forms which are more toxic to the environment and to living beings.

In Escherichia coli, the ars operon, named arsR DABC, was isolated from the plasmid R773 of the bacteria and consists of five genes (CHEN et al., 1986). The arsR gene encodes an inducible repressor the arsD is a co-repressor protein, which controls high levels of transcription. The arsA and arsB genes encode an ATPase and an efflux pump present in the cellular membrane, respectively. The arsenate reductase enzyme is encoded by the arsC gene.

It should be noted that sites polluted with arsenic usually present contamination with other heavy metals. Therefore, bacteria resistant to several heavy metal ions may be useful when used in bioremediation.

Cupriavidus metallidurans CH34 is a bacterium adapted to environments containing high concentrations of metal ions (MERGEAY et al., 2003). C. metallidurans CH34, formerly called Wautersia metallidurans CH34, Ralstonia metallidurans CH34, Ralstonia eutropha CH34, and Alcaligenes eutrophus CH34, is a β-proteobacteria, Gram-negative, non-pathogenic, firstly isolated in zinc settling ponds sediment in Liege, Belgium. It can grow in high concentrations of different heavy metals ions and radioisotopes, among them, copper (Cu²⁺); lead Pb²⁺); chromate CrO₄²⁻; cobalt (Co²⁺); nickel (Ni²⁺), zinc (Zn²⁺); bismuth (Bi³⁺), gadolinium (Gd³⁺), gold (Au⁺), silver (Au⁺), selenide (SeO₃²⁻), thallium (Tl⁺) and uranium (U²⁺).

C. metallidurans CH34 resistance to toxic metal ions is provided by a wide diversity of genes present in its four replicons: chromosome 1 (3.9 Mb), chromosome 2 (2.6 Mb) and the two large plasmids pMOL30 (234 Kb) and pMOL28 (171 KB) (MERGEAY et al., 2003). Such characteristics make this bacterium a model for studying the resistance mechanisms to heavy metals and bacteria of the main choice for biotechnological applications aimed at the recovery of environments contaminated with toxic heavy metals. The genome of this micro-organism was completely sequenced by the Joint Genome Institute, California-USA and the results are available in the database of the National Center for Biotechnology Information (NCBl).

Recent literature data show that C. metallidurans CH34 has seven ars genes located in chromosome 1. Such arsenite/arsenate resistance operon comprises the following genes: the arsR gene coding for a transcriptional regulatory protein, arsI for a protein of the glyoxalase family; arsC₁ and arsC₂ for two arsenate reductases; arsB for an arsenite efflux pump belonging to the class of ACR3 permeases; arsH for a NADPH-dependent FMN reductase, and arsP for a putative permease of “the major facilitator family” (MFS). However, the detailed operation of the C. metallidurans CH34 chromosome 1 ars operon has not yet been fully elucidated (ZHANG et al., 2009).

With the exception of Au⁺, Gd³⁺ and SeO₃²⁻, which are intracellularly precipitated, the fantastic cellular protection network presented by the bacterium C. metallidurans CH34 detoxifies its cytoplasm, but not the environment. In the case of arsenic ions, detoxification occurs probably by efflux. Therefore, this bacterium in its natural state cannot meet the desirable characteristics to be used in environmental bioremediation strategies against arsenic ions, but represents an excellent microorganism that offers potential to receive genetic improvements aiming at biotechnological applications.

The use of natural surface proteins as a tool for anchoring heterologous proteins in the so called “cell surface display” systems has presented a broad application in different scientific areas. Through this strategy, several peptides were anchored on the surface of different bacteria with various purposes, such as antibody production, biocatalysis, bioremediaton among others (WERNERUS; STAHL, 2004).

In the case of bioremediation, the literature has recently shown that recombinant microorganisms, whose cell surface has been enriched with metal chelating proteins, have higher capacity for metal ion adsorption when compared to the non-recombinant strain, therefore representing a biotechnological strategy for the development of high potential bioremediator agents.

Recent studies have revealed various strategies that may be used to anchor peptides on the external membrane of Gram-negative bacteria: gene insertions in the coding sequences of cellular structures such as flagella, pili, external membrane proteins, or even using the mechanism of self-carrier proteins secretion.

Klauser and his collaborators (KLAUSER; POHLNER; MEYER, 1990) were the first to use as a tool for peptides anchoring, an adaptation of the natural secretion system of the N. gonorrhoeae IgA protease for its anchoring on the surface of other bacteria. Said researchers used parts of the IgA protease secretion system for the anchoring the β domain of the cholera toxin (CtxB) on the Salmonella typhimurium cell surface. To do so, the gene sequence corresponding to the CtxB domain was cloned between the coding sequences of the signal peptide (PS) and β-domain secretion system of the N. gonorrhoeae IgA protease, and after the construction expression, these authors found that the CtxB peptide was exposed on the microorganism cell surface.

From then on, various peptides were anchored in the external membrane of Gram-negative bacteria (E. coli, C. metallidurans, N. gonorrhoeae, N. meningitidis, S. typhimurium, P. putida) through this system including a mouse metallothionein in the C. metallidurans CH34 external membrane (WERNÉRUS; STAHL, 2004).

In a recent work developed by our group, the same mechanism of the N. gonorrhoeae IgA protease secretion was used to anchor the synthetic phytochelatin EC20 in the external membrane of C. metallidurans CH34, proving to be an appropriate strategy for anchoring a desired recombinant protein to the bacteria cell surface (PI0801282-2).

The anchorage of polypeptides of high affinity to metal ions in the bacterial wall generally comprises peptides rich in cysteines. Frequently used polypeptides are the metallothioneins, natural or synthetic phytochelatins, and glutathione. The EC20 synthetic phytochelatin, for example, shows high ability to immobilize a wide variety of heavy metals from the external environment, however, since it has a very large number of cysteines positioned in the primary structure, these peptides do not feature selectivity, making it impractical to use them in the removal and recycling of specific ions.

On the other hand, the regulatory ArsR protein encoded by the ars operon of Gram-negative bacteria is a dimeric protein which is conserved in bacterial species. This protein is considered to be the arsenic ions ligand of higher affinity and specificity already reported (ZHANG et al., 2009). Nevertheless, there are no published data which show the expression and anchoring of the ArsR protein on the cell surface of microorganisms.

The ArsR protein structure and its binding motif to the arsenic ions are still little known. Crystallographic studies of the Escherichia coli ArsR protein show a trigonal pyramid and hypothesize a site responsible for binding the protein to the metalloid trivalent form. The interaction would occur due to the presence of three cysteine residues located in the N-terminal portion of (Cys32, Cys34, Cys37) the molecule in a α-helix region. The simultaneous interaction of the inorganic arsenic with Cys32 and Cys34 residues would result in abnormal association, since the reason suggested would cause a significant proteic structural disruption. Therefore, the structural conformation of the ArsR protein has not been completely explained and further studies need to be performed.

The ArsR protein of C. metallidurans contains 109 amino acids and the binding site with the metalloid comprises the CCXGXXC motif located on the molecule C-terminal portion (ZHANG et al., 2009).

Considering that inorganic arsenic is one of the most toxic substances and is still released in nature in large quantities by human activities worldwide, the need for the construction of bacteria especially designed for arsenic ions bioremediation is justified.

Hence, the present invention describes the use of a “cell surface display” strategy to enrich the surface of Gram-negative bacteria with the C. metallidurans CH34 ArsR protein, which has a high capacity of specific binding to arsenic ions, for application in bioremediation processes.

In 2008, our research group filed the patent application PI0801282-2 which describes the construction of a genetically modified C. metallidurans CH34 lineage to express the EC20 protein on its cell surface. This lineage presents increased ability to bind toxic metals ions on the cell surface. To obtain this recombinant lineage, the inventors have provided the C. metallidurans CH34 bacterium with a genetic system which allowed the anchoring of the EC20 protein on its surface. Such genetic system was constructed in vitro using the coding sequences of the signal peptide and the anchoring domain of the Neisseria gonorrhoeae IgA protease secretion system, and the whole gene fusion (gene system) was expressed under the translational control of the pan promoter derived from Bacillus subtilis (RIBEIRO-DOS-SANTOS, et al., 2010).

However, due to the large number of cysteine residues in the polypeptidic chain of the synthetic phytochelatin EC20 and high capacity for heavy metals in general to bind tightly to the sulfhydryl groups (—SH) of these amino acids, EC20 does not show selectivity for capturing metal ions, therefore, systems employing specific and selective binding molecules with high affinity to certain ions become necessary, since the environmental contamination can occur owing to the presence of a specific ion in the ecosystem.

Thus, at a subsequent time, the gene encoding the synthetic phytochelatin EC20, previously inserted in the pCM2 plasmid, was replaced by the gene encoding the protein MerR, of the C. metallidurans CH34 mer operon, which has high affinity and specificity in the capture of mercury. The new plasmid, called pCM-Hg, was inserted into the Gram-negative bacteria E. coli and C. metallidurans CH34. With this strategy it was possible to enhance the cellular surface of these bacteria by means of expressing and anchoring the C. metallidurans CH34 MerR protein using the secretion mechanism of the N. gonorrhoeae IgA protease and the pan promoter. As a result, we obtained recombinant Gram-negative bacteria with superior ability to specifically adsorb mercury ions, which may be used in bioremediation process in mercury contamination vases. This invention led to the filing of patent application PI 1101557-8, on Apr. 29, 2011.

However, the above invention is specifically directed to bioremediation in cases of mercury contamination, thus there remains a need for a solution of the bioremediation of waste water contaminated with arsenic.

Such need led to the present invention, whose proposed technical solution involves: 1) construction of a recombinant plasmid containing the structural sequence of the arsR gene of C. metallidurans CH34 chromosome 1 fused to the gene cassette for the expression and anchoring of heterologous proteins under the regulation of the pan promoter; 2) insertion of this recombinant plasmid in C. metallidurans CH34 and E. coli UT5600 bacteria; 3) construction of a new recombinant bacterium that can be successfully used for adsorption of As⁵⁺ ions. Therefore, the approach hereby presented allows for arsenic ions removal by means of recombinant Gram-negative bacterial lineages, constructed as disclosed in the present description.

### BRIEF DESCRIPTION OF THE INVENTION

The purpose of the present invention is the construction of a recombinant plasmid containing a gene sequence which, when expressed, allows the anchorage of a chelating protein of metal ions, more specifically, of arsenate ions (As⁵⁺) on the cellular surface of Gram-negative bacteria, such as C. metallidurans CH34 and E. coli UT5600. It should be noted, nevertheless, that the peptide in question also has high affinity and specificity to bind to the trivalent arsenic form (As³⁺) (ZHANG et al., 2009).

Bacterial Gram-negative lineages containing said recombinant plasmid for arsenic ions adsorption and their potential use in environmental bioremediation processes are also objects of the present invention.

Furthermore, the invention provides an arsR gene with modifications.

It is an additional object of the present invention the attainment of a specific expression vector containing a gene cassette with a signal peptide coding sequence.

Moreover, the present invention provides a recombinant plasmid pCM-As carrying the arsR anchoring cassette.

The present invention discloses recombinant strains containing the recombinant plasmid pCM-As, which derive from certain Gram-negative bacteria.

The present invention provides a recombinant plasmid pCM-As carrying a genetic construct that confers bacterial resistance to arsenic ions.

The present invention reports the use of a recombinant plasmid pCM-As in other Gram-negative bacteria to provide new recombinant strains suitable for arsenic bioremediation.

The present invention is intended to describe the construction of recombinant Gram-negative bacteria with increased potential to carry out the decontamination of waters and environments containing inorganic arsenic ions.

### DESCRIPTION OF FIGURES

FIG. 1 shows the steps for obtaining the chromosome 1 arsR gene (GeneID: 4037120) of C. metallidurans CH34 wild type strain, devoid of the TGA stop codon: FIG. 1A (Panel A) shows the migration in agarose gel of total C. metallidurans CH34 DNA previously extracted, which was used as a template DNA to obtain the arsR gene, present on chromosome 1, by employing Polymerase chain reaction amplification of DNA (PCR), which was performed. FIG. 1B (panel B) shows the fragment of 342 base pairs (bp) obtained by PCR, corresponding to the arsR gene of C. metallidurans CH34 chromosome 1, without the termination codon.

FIG. 2 (panel A) shows the representative scheme of the C. metallidurans CH34 arsR gene cloning into an intermediate plasmid vector, pGEM-T (Promega®), resulting in the pGEMT-As plasmid (3342 bp): FIG. 2A (panel A) shows the insertion of the arsR gene obtained by PCR (342 bp) into the pGEM-T plasmid vector (3,000 bp). FIG. 2B (panel B) shows the analysis of the pGEMT-As plasmid by restriction enzyme digestion and agarose gel electrophoresis, confirming the construction.

FIG. 3 shows the representative scheme of the pCM-As plasmid construction: the pCM-Hg plasmid (6,937 bp), previously constructed in our laboratory, which contains an expression-anchorage cassette comprising the coding sequence of the β-domain of the N. gonorrhoeae IgA protease secretion system (1,332 bp) and the merR gene (453 bp) inserted between the gene sequences of the signal peptide (51 bp) and E-tag antigen (36 bp), under control of the pan promoter (PI1101557-8), was digested with XbaI and SalI restriction enzymes. Upon digestion, the merR gene was released from pCM-Hg, and the resulting plasmid was denominated pCM (6,490 bp) (SEQ ID No 4). The DNA fragment of 342 bp corresponding to the arsR gene of C. metallidurans CH34, also endowed with XbaI and SalI cohesive ends, was inserted into pCM (6,490 bp) (SEQ ID No 4), giving rise to the pCM-As plasmid of 6,832 bp (SEQ ID No 5).

FIG. 4 shows the analysis of total protein extraction visualized by 15% SDS-PAGE and “Coomassie Blue R250” staining. FIG. 4A (Panel A): Total proteins from E. coli UT5600 and recombinant E. coli UT5600/pCM-As. FIG. 4B (Panel B): Total proteins from C. metallidurans CH34 and recombinant C. metallidurans CH34/pCM-As. The arrows indicate the expression of the ArsR-E-tag-R-domain fusion protein (58 kDa) by the recombinant bacteria.

FIG. 5 shows the micrographs of Immunofluorescence Microscopy (1,000× magnification) of wild type and recombinant C. metallidurans and E. coli cells. Cells were incubated with mouse anti-E-tag primary antibody (GE Life Sciences) and fluorescently stained with anti-mouse secondary FITC-conjugated antibody (Sigma-Aldrich). The expression of the ArsR-E-tag-β-domain fusion protein (58 kDa) on the recombinant cells surface was confirmed (Panels B and D). 5A—C. metallidurans CH34; 5B—recombinant C. metallidurans CH34/pCM-As; 5C—E. coli UT5600; and 5D—recombinant E. coli UT5600/pCM-As.

FIG. 6 shows the cell fractionation of wild type and recombinant E. coli cells: protein extracts from E. coli UT5600 and E. coli UT5600/pCM-As were fractionated in Soluble Fraction (SF), Internal Membrane (IM), and External Membrane (EM). Panel 6A: protein fractions were visualized by SDS-PAGE and “Coomassie Blue R250” staining. The arrow indicates the expression of the ArsR-E-tag-β-domain fusion protein (58 kDa) on the EM of the recombinant E. coli UT5600/pCM-As. Panel 6B: the expression of the ArsR-E-tag-β-domain fusion protein (58 kDa) on the EM of the recombinant E. coli UT5600/pCM-As cells was confirmed by Western Blotting using anti-E-tag primary antibody (GE Life Sciences) and peroxidase conjugated antibody (Sigma-Aldrich).

FIG. 7 shows the cell fractionation of wild type and recombinant C. metallidurans CH34 cells: protein extracts from C. metallidurans CH34 and C. metallidurans CH34/pCM-As were fractionated in Soluble Fraction (SF), Internal Membrane (IM) and External Membrane (EM). Panel 7A: protein fractions were visualized by 15% SDS-PAGE and “Coomassie Blue R250” staining. The arrow indicates the expression of the ArsR-E-tag-β-domain fusion protein (58 kDa) on the EM of the recombinant C. metallidurans CH34/pCM-As cells. Panel 7B: the expression of the ArsR-E-tag-β-domain fusion protein (58 kDa) on the EM of the recombinant C. metallidurans CH34/pCM-As cells was confirmed by Western Blotting using anti-E-tag primary antibody (GE Life Sciences) and peroxidase conjugated antibody (Sigma-Aldrich).

FIG. 8 shows micrographs obtained by Transmission Electron Microscopy (TEM) of wild type and recombinant C. metallidurans CH34 cells (40,000× magnification). Cells were incubated in sterile ultrapure water (Milli-Q) or in sterile ultrapure water solutions (Milli-Q) containing 500 mM of sodium arsenate (Na₃As0₄) for 2 hours. Panel 8A shows wild type C. metallidurans CH34 cells after incubation in water. Panel 8B shows wild type C. metallidurans CH34 cells after incubation in 500 mM Na₃As0₄. Panel 8C shows C. metallidurans CH34/pCM-As recombinant cells after incubation in water. Panel 8D shows C. metallidurans CH34/pCM-As recombinant cells after incubation in 500 mM Na₃As0₄. Red arrows indicate the metalloid accumulation onto the cellular surface of the recombinant bacteria. Blue arrows indicate cytoplasmic accumulation.

FIG. 9 shows micrographs obtained by Transmission Electron Microscopy (TEM) of wild type and recombinant E. coli cells (40,000× magnification). Cells were incubated in sterile ultrapure water (Milli-Q) or in sterile ultrapure water solutions (Milli-Q) containing 500 mM of sodium arsenate (Na₃As0₄) for 2 hours. Panel 9A shows wild type E. coli UT5600 cells after incubation in water. Panel 9B shows wild type E. coli UT5600 cells after incubation in 500 mM Na₃As0₄. Panel 9C shows the recombinant E. coli UT5600/pCM-As cells after incubation in water. Panel 9D shows the recombinant E. coli UT5600/pCM-As cells after incubation in 500 mM Na₃As0₄. Blue arrows indicate metalloid accumulation onto the cellular surface of the recombinant bacteria. Red arrows indicate cytoplasmic accumulation.

FIG. 10 shows the Minimal Inhibitory Concentration (MIC) of E. coli UT5600 wild type cells (Panel A) and recombinant E. coli UT5600/pCM-As cells (Panel B). Panel C illustrates the comparison between the growth levels of E. coli wild type and recombinant cells in the presence of different concentrations of Na₃As0₄ ranging from 0-50 mM. After incubation at 28° C. for 48 h, the bacterial growth was measured by reading the absorbance at 600 nm (OD600) in a spectrophotometer.

FIG. 11 shows the Minimal Inhibitory Concentration (MIC) of C. metallidurans CH34 wild type cells (Panel A) and recombinant C. metallidurans CH34/pCM-As cells (Panel B). Panel C shows the comparison between the growth levels of C. metallidurans CH34 wild type and recombinant cells in the presence of different concentrations of Na₃As0₄ ranging from 0-1,000 mM. After incubation at 28° C. for 48 h, the bacterial growth was measured by reading the absorbance at 600 nm (OD600) in a spectrophotometer.

FIG. 12 shows the As⁵⁺ ions adsorption by C. metallidurans CH34 wild type and recombinant cells after incubation in 1 mM Na₃As0₄ for different times (0, 10, 30, 60, 120 and 240 min). The pentavalent arsenic concentration in the cells is indicated in μg of As⁵⁺ per gram of bacterial dry mass (ppm).

FIG. 13 shows the As⁵⁺ ions adsorption by E. coli UT5600 wild type and recombinant cells after incubation in 1 mM Na₃As0₄ for different times (0, 10, 30, 60, 120 and 240 min). The pentavalent arsenic concentration in the cells is indicated in μg of As⁵⁺ per gram of bacterial dry mass (ppm).

FIG. 14 shows the comparison of the As⁵⁺ ions adsorption efficiency by C. metallidurans CH34/pCM-As and E. coli UT5600/pCM-As recombinant strains (micrograms of As⁵⁺ per gram of bacterial dry mass) after incubation in 1 mM Na₃As0₄ for different times.

### DETAILED DESCRIPTION OF THE INVENTION

The present invention describes the construction of a recombinant plasmid containing a gene sequence which, when expressed, allows the anchorage of a chelating protein of metal ions, more specifically of inorganic arsenic, on the cellular surface of Gram-negative bacteria. DNA and bacterial cells manipulations were carried out following protocols.

The DNA fragment corresponding to the arsR gene (342 bp) without the termination codon (SEQ. ID No 1) was amplified by PCR from the total DNA of C. metallidurans CH34 (ATCC®-43123TM).

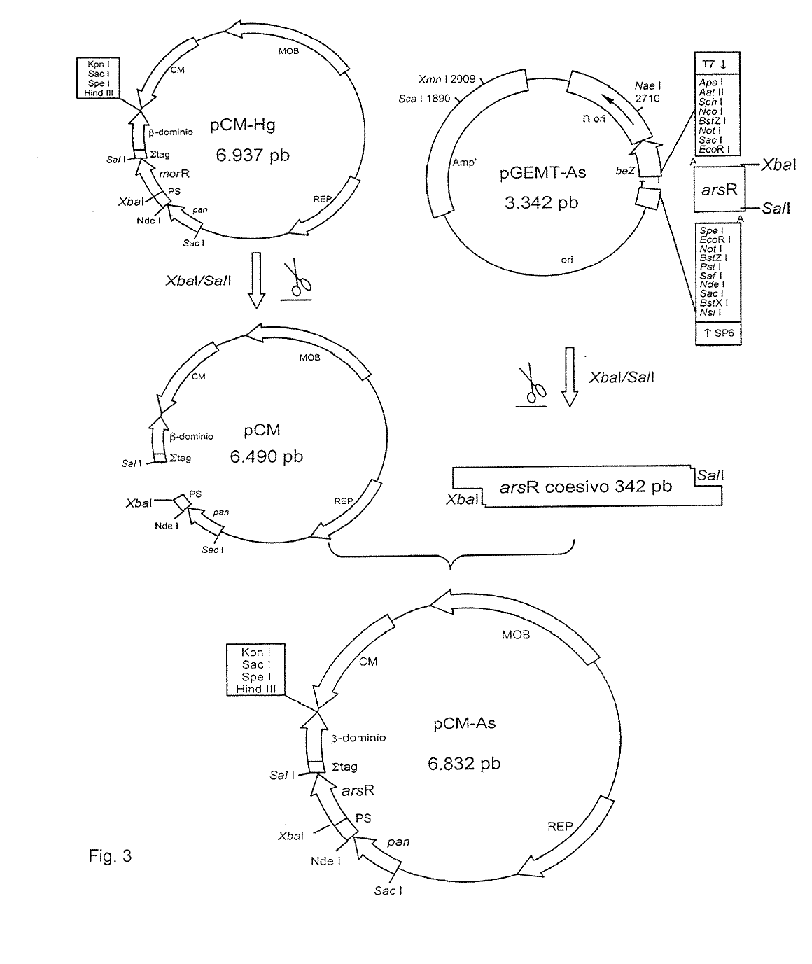

The arsR fragment was inserted into the pCM plasmid (SEQ. ID No ° 4), originated from the pCM-Hg of 6,937 bp (PI1101557-8) (FIG. 3) between the coding sequences of the signal peptide (PS) of 51 bp and E-tag reporter epitope (36 bp), followed by the coding sequence of the (3-domain of the Neisseria gonorrhoeae (1,332 bp) IgA protease secretion system, resulting in the pCM-As plasmid (SEQ ID No 5). All PS-arsR-E-tag-β-domain gene fusion fell under pan promotor control (427 bp), derived from Bacillus subtilis (SEQ. ID N° 3).

The pCM-As plasmid was inserted in C. metallidurans CH34 cells (wild type strain isolated from sediments in zinc settling ponds in Liege, Belgium by genetic transformation, yielding the recombinant strain C. metallidurans CH34/pCM-As.

The pCM-As plasmid was inserted in E. coli UT5600 cells (Commercial Lineage 1—Promega®), stored at the Laboratory of Genetics of Microorganisms, Department of Microbiology, University of Sao Paulo, by genetic transformation, yielding the recombinant strain E. coli UT5600/pCM-As.

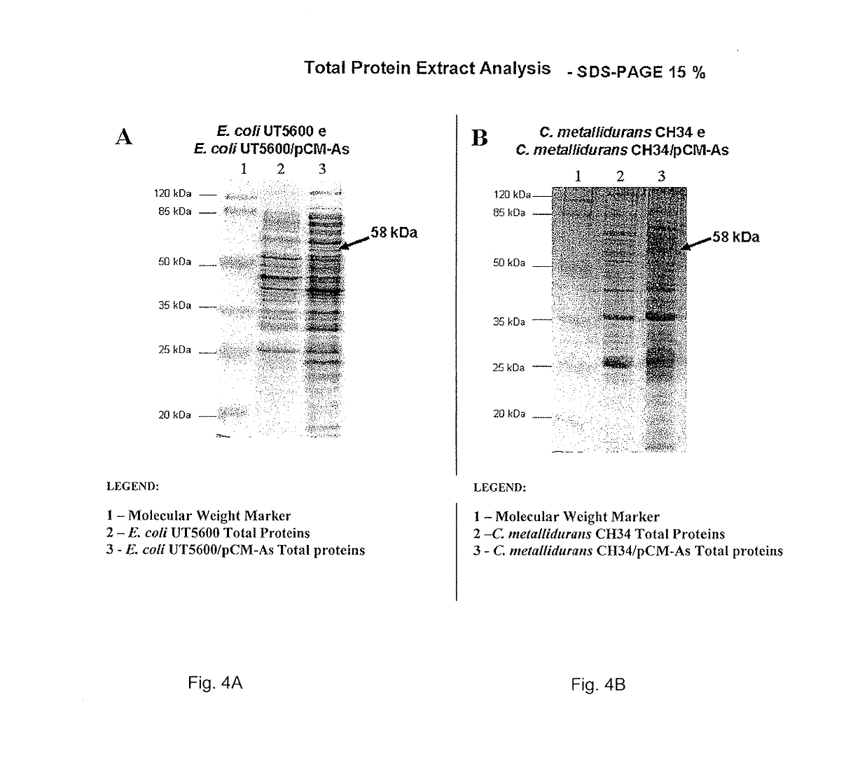

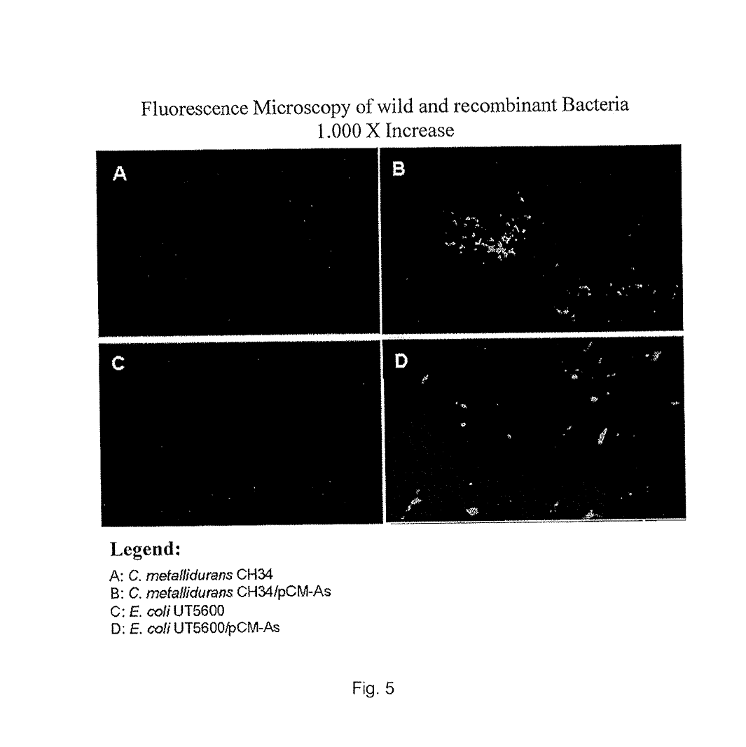

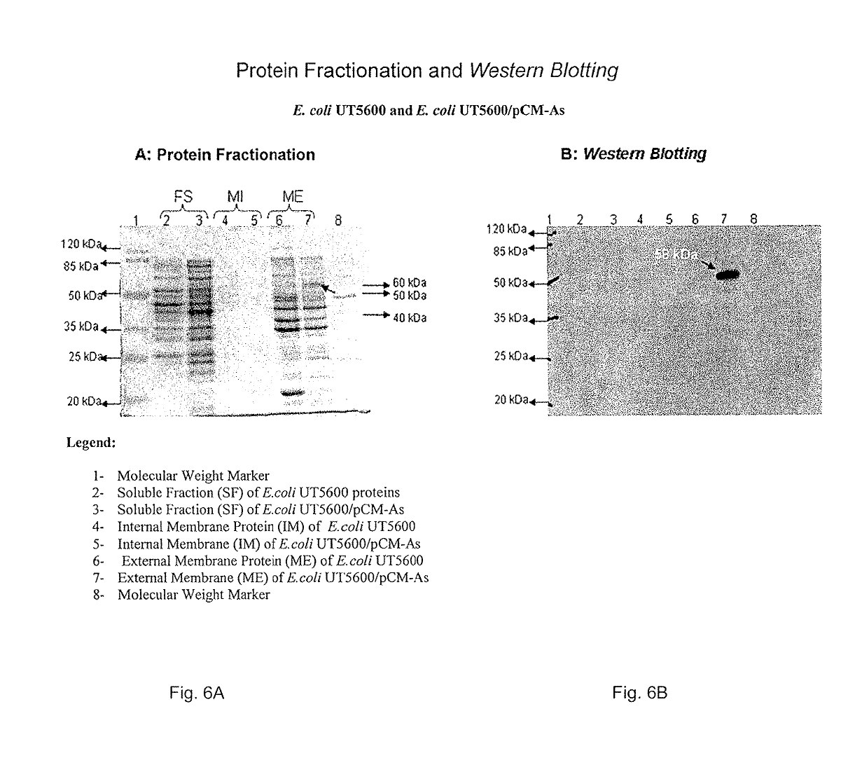

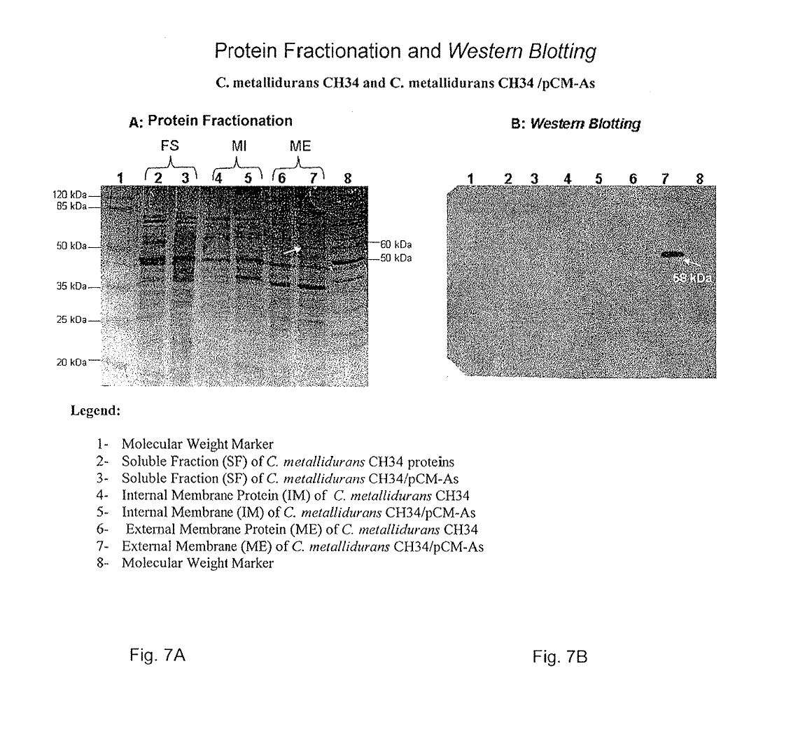

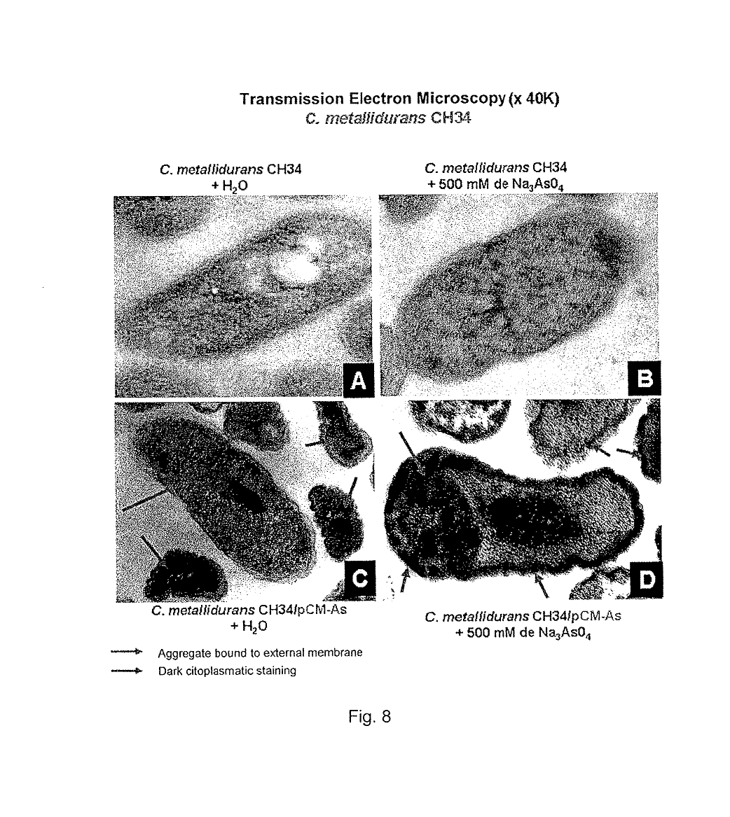

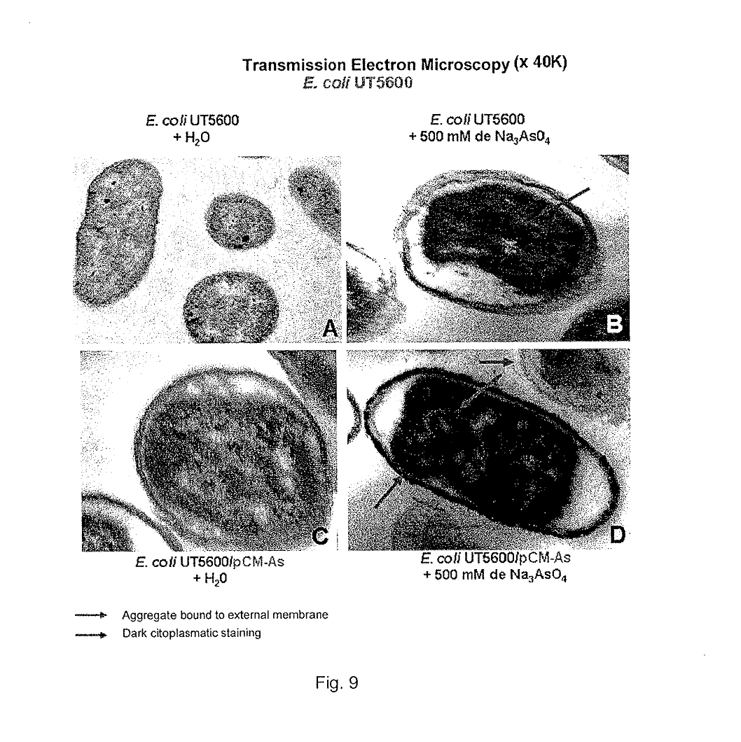

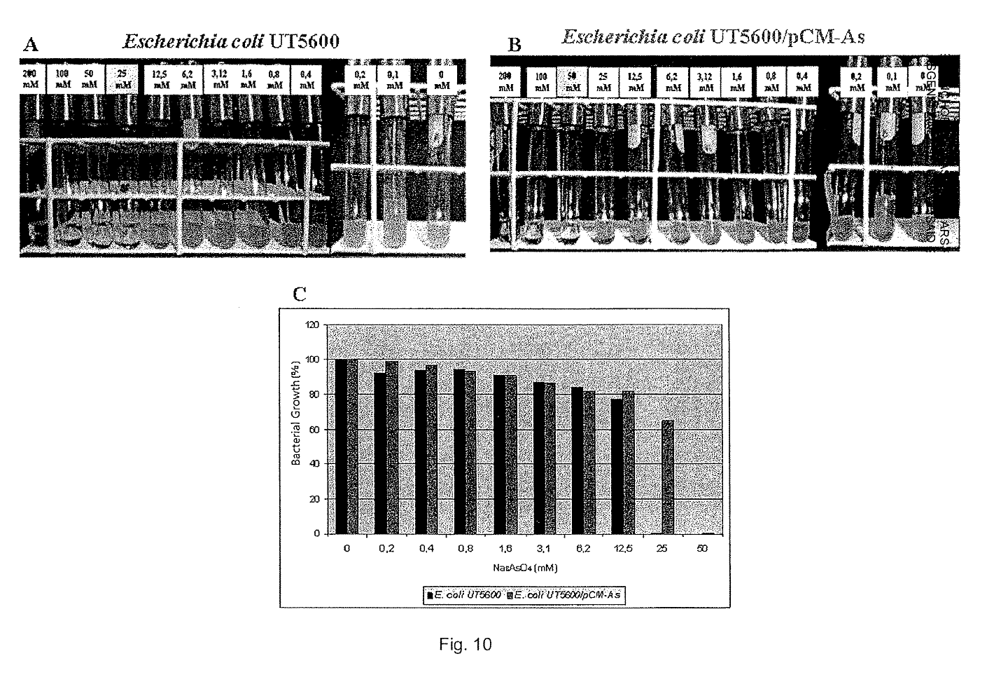

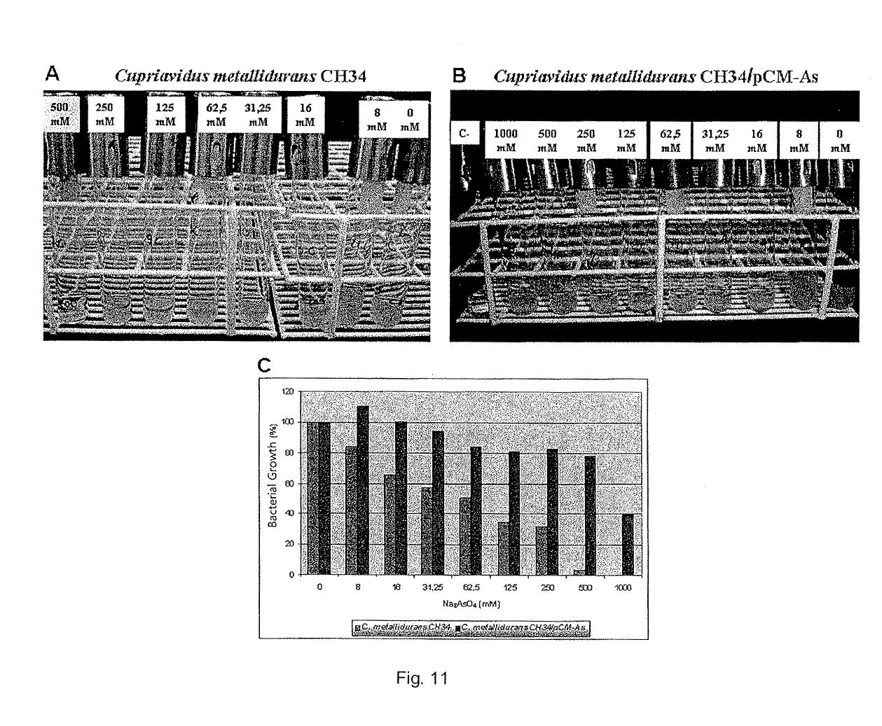

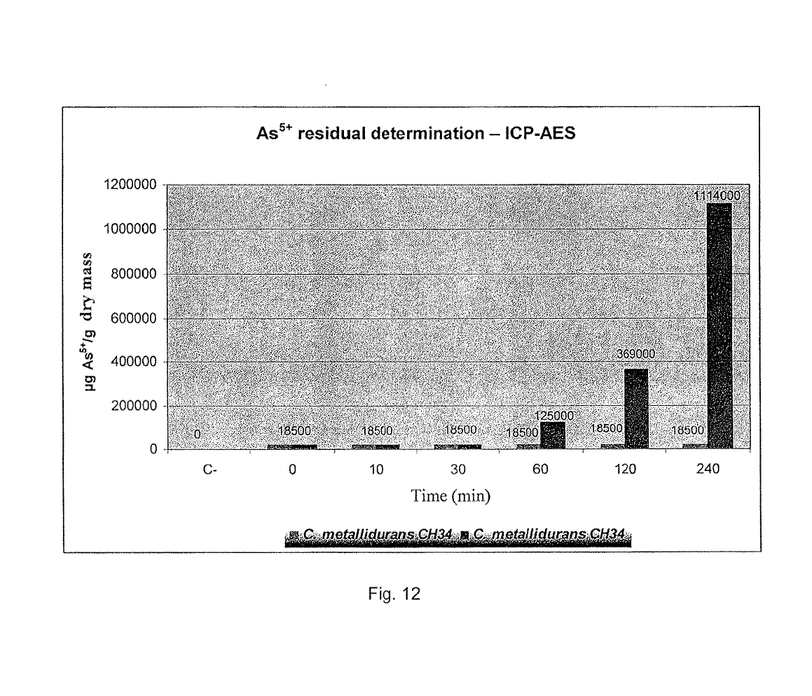

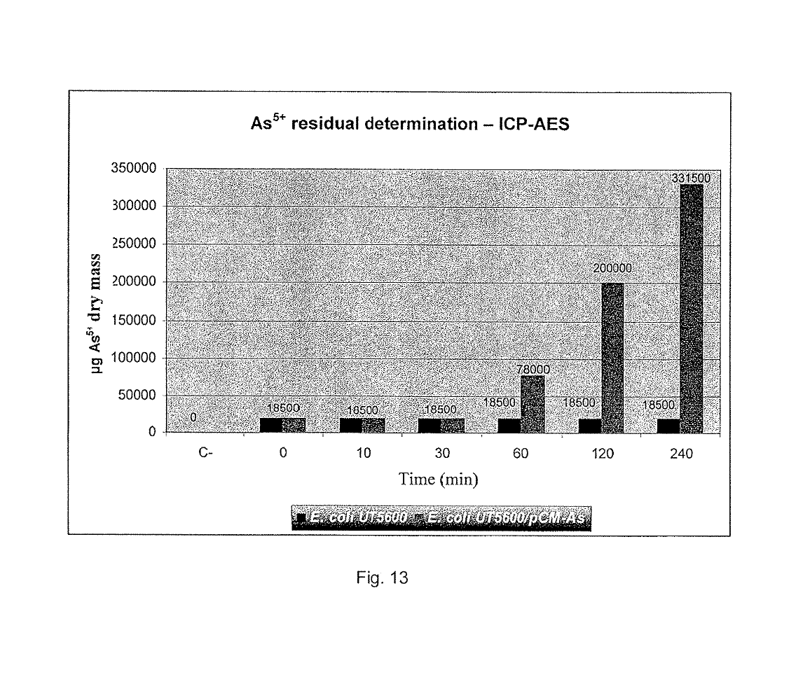

The recombinant C. metallidurans CH34/pCM-As and E. coli UT5600/pCM-As cells produce the ArsR protein anchored on their cellular surfaces, as confirmed by several techniques: 1) total protein extraction profiles observed by SDS-PAGE (FIG. 4); 2) fluorescence microscopy using the anti E-tag antibody (GE life Sciences), since the E-tag antigen is expressed fused to the ArsR protein (FIG. 5); 3) protein profiles of subcellular fractions visualized by SDS-PAGE with the respective “Western blotting” immunoassay to identify the protein of interest (FIGS. 6 and 7). These new recombinant bacteria demonstrated the expression and anchoring of the C. metallidurans CH34 ArsR protein. Additionally, it was found that recombinant cells carrying the pCM-As plasmid show increased capacity of As⁵⁺ ions adsorption on their cellular surfaces, as verified by Transmission Electron Microscopy (FIGS. 8 and 9). The pCM-As plasmid conferred to these new recombinant bacteria increased resistance (an increase greater than or equal to 100%) to the toxic effects of arsenate ions (As⁵⁺) (FIGS. 10 and 11). The patent application especially refers to the transgenic strains of Cupriavidus metallidurans CH34 and Escherichia coli UT5600 containing the recombinant pCM-As plasmid, which were capable of removing pentavalent arsenic ions from the external environment in significantly higher concentrations when compared to the control strains due to the presence of the ArsR protein on their cellular surface (FIGS. 12 and 13).

The present application provides Gram-negative bacterial strains containing said recombinant plasmid for potential use for As⁵⁺ adsorption and application in environmental bioremediation processes.

In a first embodiment, the present invention provides an arsR gene obtained in vitro without the protein synthesis stop codon SEQ. ID No 1.

In a second embodiment, the present application consists in obtaining a recombinant plasmid containing the arsR gene with modifications, yielding the pGEMT-As plasmid (SEQ. ID N° 2).

In a third embodiment, the present invention provides the construction of a plasmid containing a gene fusion comprising the coding sequence of a signal peptide, the coding sequence of the arsR gene, the coding sequence of an E-tag epitope, the coding sequence of the IgA protease β-domain. This 2,233 bp fragment allows the expression and cell surface display (anchorage) of the ArsR protein of C. metallidurans CH34 (SEQ. ID No 3).

In a fourth embodiment, the invention provides a pCM-As recombinant plasmid carrier of the arsR anchorage cassette under the expression control of the Bacillus subtilis pan promoter.

In addition, the patent application relates to transgenic strains deriving from Escherichia coli and Cupriavidus metallidurans, as well as other Gram-negative bacteria besides those above mentioned, containing the recombinant pCM-As plasmid, which may be microorganisms with the potential to be used in the removal of inorganic arsenic ions from contaminated environments due to the expression of the ArsR protein anchored to their cellular surface.

The patent application aims to develop recombinant strains of Gram-negative bacteria with potential for decontamination of environments containing arsenic. The genetic modification introduced in these lineages confers to them the capacity to produce an As⁵⁺ chelating protein of higher affinity (ArsR), and then secrete this protein through the inner and outer membrane, with the protein being finally anchored in the external membrane of the cells. These bacteria, now covered by ArsR protein molecules, can act as a magnet for As⁵⁺ ions and can be applied to new remediation processes. In a subsequent step, adsorbed metals can be recovered by desorption for reutilization, or disposed by incineration of the bacteria.

The present application provides a recombinant plasmid with an additional ability to increase survival levels for Gram-negative bacteria in an environment contaminated with As⁵⁺ ions, and its use in Gram-negative bacteria sensitive to this metalloid to provide bioremediation capacity in Gram-negative cells considered impracticable for this application.

The present invention consists in the construction of Gram-negative bacteria recombinant strains with the outer membrane enriched by the ArsR protein, such bacteria to be used in bioremediation processes of the most toxic arsenic forms. The various steps of DNA manipulation and amplification, bacterial genetic transformation, DNA and protein purification and analysis, and enzyme immunoassays were performed.

For that end, the arsR gene (342 bp) was amplified from total DNA of the wild type C. metallidurans CH34 bacterium by PCR. The obtained DNA amplicon was inserted into the pGEM-T cloning vector (Promega®), giving rise to the pGEMT-As plasmid. The pGEMT-As plasmid was inserted in the host E. coli DH5α by genetic transformation. This recombinant plasmid was isolated from selected transformants (white colonies) and subjected to enzymatic digestion with XbaI/SalI and for arsR gene release with specific cohesive ends.

The arsR gene with cohesive ends was inserted into the pCM plasmid (SEQ ID No 4), previously digested with the same restriction enzymes. The pCM plasmid derives from the pCM-Hg plasmid (PI1101557-8), which originated from the pCM2 plasmid (PI 0801282-2).

The pCM plasmid is suitable for heterologous proteins expression and anchoring in C. metallidurans and E. coli, as well as other Gram-negative bacteria. The pCM-As plasmid (FIG. 3) contains: a) the Bacillus subtilis pan promoter, which is able to drive the expression of high levels of recombinant proteins in E. coli and in C. metallidurans without the need of addition of any inducers. Furthermore, protein expression under control of the pan promoter is increased upon incubation of the C. metallidurans CH34 cells in the presence of metal ions; b) the full anchorage cassette for the expression of a desired protein on the cellular surface of Gram-negative bacteria; c) the E-tag sequence allowing immunoassays. Thus, the pCM-As plasmid (SEQ ID N° 5) derives from the pCM-Hg expression plasmid, which was previously developed by the authors of this invention (PI1101557-8).

After merR gene removal from the pCM-Hg plasmid, the arsR gene was inserted thereon, resulting in the recombinant pCM-As plasmid, genetic transformation vector of the present invention. The pCM-As plasmid was inserted in the E. coli DH5α bacterium (Promega®, stored in the Laboratory of Genetics, Department of Microbiology, University of Sao Paulo. The construction of the recombinant PCM-As plasmid was confirmed by restriction analysis and DNA sequencing.

Upon confirmation of the plasmid PCM-As construction, said PCM-As was introduced into the Gram-negative bacteria E. coli UT5600 (Promega®), and C. metallidurans CH34 (wild lineage isolated from sediments in zinc settling tanks in Liege, Belgium by means of bacterial genetic transformation. Cells of such lineages, non-transformed and recombinant, being the latter hosts of the pCM-As plasmid, were grown in the absence of any added inducer and the ARS-R anchorage cassette expression was confirmed by comparing the protein profiles of each lineage by SDS-PAGE 15%. As the secretion β-domain is 45 kDa, the E-tag epitope is 1.4 kDa, and the ArsR protein of C. metallidurans CH34 is 11.4 kDa, these residues together form a hybrid protein of 58 kDa. The electrophoretical analysis of total proteins extracted from each lineage allowed the confirmation that the recombinant strains present an extra band of the expected size (58 kDa), when compared to the protein profiles of non-recombinant strains.

The functionality analysis of the anchoring system in recombinant C. metallidurans CH34/pCM-As and E. coli UT5600/pCM-As bacteria was carried out by fluorescence microscopy, incubating the cells with primary anti-E-tag antibody produced in mice (GE Life Sciences) and FITC-conjugated anti-mouse secondary antibody for fluorescence emission (Sigma-Aldrich). This assay resulted in the observation of fluorescent green signal emitted after specific recognition reaction between antigen and antibody, allowing the confirmation that the E-tag epitope is efficiently transported to the outer membrane of both recombinant cells. Non transformed lineages (no pCM-As plasmid) were used as negative controls of the experiment and showed no reactivity.

In order to investigate ArsR protein anchorage in the outer membrane of recombinant bacteria, the cellular proteins were fractionated into soluble fraction (SF), inner membrane (IM) and external membrane (EM). The three obtained fractions for each strain were visualized by Sodium Dodecyl Sulfate Polyacrylamide Gel Electrophoresis (SDS-PAGE). After electrophoretic analysis, the protein fractions were transferred to a nitrocellulose membrane and the expression of the ArsR/E-tag/β-domain fusion in the external membrane of recombinant bacteria was confirmed using the E-tag epitope as a reporter, which is specifically recognized by the anti-E-tag antibody (commercial primary antibody produced in mice, GE Life Sciences) in enzyme immunoassays. The corresponding wild type strains were used as negative controls of the experiment.

A reactive band of 58 kDA was visualized only in the EM fraction of recombinant strains, which demonstrates the expression of the ArsR/E-tag/8-domain fusion on the cell surface. No reactivity was found in the soluble or inner membrane fractions. Also, our results demonstrate that the heterologous protein was successfully produced by the cells and that the secretion-anchoring mechanism was functional. Such results are in agreement with those obtained by VEIGA et al. (2002) who used this secretion mechanism for peptide anchoring on E. coli UT5600 outer cell surface. It is therefore concluded that the construction of genetically modified E. coli UT5600 and C. metallidurans CH34 Gram-negative bacteria, which contain the outer membrane enriched with the ArsR protein, was successfully performed.

To observe the ability to bind arsenic ions in the external membrane, recombinant cells carrying the pCM-As plasmid were incubated in 500 mM sodium arsenate (Na₃AsOH₄) and visualized by Transmission Electron Microscopy (TEM). In cells of both recombinantstrains, the formation of aggregates attached to the external membrane showing the accumulation of arsenate ions on the cellular surface was observed. This indicates that, indeed, the As⁵⁺ ions are being captured by the recombinant protein and that the presence of ArsR protein anchored on the cells surface has enhanced the bioremediator ability of the constructed lineages. When cultured in Na₃AsOH₄, either wild type or reombinant cells showed dark cytoplasmic staining, indicating that, in the presence of the metalloid, ars operon genes transcription takes place, activating the natural system of bacterial detoxification, resulting in the precipitation of intracellular As⁵⁺.

The recombinant bacteria developed in the present invention have enhanced ability to adsorb As⁵⁺ ions, enabling the metalloid recovery by desorption. Arsenic precipitation within the cells enhances the extraction of the potentially toxic metalloid from contaminated environments, and an incineration of the bacteria used after ions recovery may be simply employed.

Many publications have focused on the cytoplasmic overexpression of the arsR gene in recombinant bacteria for possible use in arsenic bioremediation processes arising from intracellular precipitation. However, such method does not provide the recovery of the metalloid by desorption, being possible only the incineration of bacteria used in these cases. ArsR expression and anchoring in microorganisms, whether Gram-positive bacteria, Gram-negative bacteria or yeast, has not been reported in the literature until now, which emphasizes the innovative nature of the present invention.

In addition, the recombinant constructed bacteria introduced herein produce the ArsR protein constitutively under the control of the Bacillus subtilis pan promoter, which proved to be able to express high levels of recombinant proteins in E. coli without artificial induction, besides promoting enhanced protein expression in C. metallidurans CH34 in the presence of metal ions (RIBEIRO-DOS-SANTOS et al.). This fact represents a major advance in terms of new bioremediation agents, since not having to add external inducers constitutes a relevant biotechnological novelty and increases the economic feasibility of biological processes for the recovery of degraded areas.

In addition to the transgenic strain E. coli UT5600/pCM-As, the present invention discloses the C. metallidurans CH34/pCM-As recombinant lineage. Given that C. metallidurans CH34 is naturally able to survive in environments highly contaminated with heavy metals (MERGEAY, 1985); the C. metallidurans CH34/pCM-As strain constructed in this invention presents itself as an industrial model to be used in bioremediation processes of waters and environments contaminated by arsenic.

As⁵⁺ ions resistance was evaluated in wild and recombinant E. coli UT5600 lineages. The Minimum Inhibitory Concentration (MIC) in growth medium containing different concentrations of Na₃As0₄ was found to be 25 mM for E. coli UT5600. The recombinant lineage carrying the pCM-As plasmid presented a MIC of 50 mM, showing increased survivability, 100% higher in relation to the wild lineage.

As⁵⁺ ions resistance of C. metallidurans CH34 and C. metallidurans CH34/pCM-As cells were also determined. The MIC against different Na₃As0₄ concentrations for C. metallidurans CH34 was 500 mM, indicating high natural resistance to arsenate. The MIC of C. metallidurans CH34 cells carrying the pCM-As plasmid was >1,000 mM, indicating an increase in survivability to As⁵⁺ ions greater than 100%. The resistance of wild type C. metallidurans CH34 and the recombinant lineage C. metallidurans CH34/pCM-As to extreme arsenic levels presented herein was first identified in this work. From such results, the bacterial strain C. metallidurans CH34/pCM-As can be regarded as the most arsenate-resistant bacterium already reported (Table 1).

Therefore, the pCM-As plasmid described in the present invention has been able to increase the capacity of cell survival of both Gram-negative bacteria which were employed as hosts. This indicates that it can be used in other Gram-negative bacteria in order to increase the survival rates of said bacteria to arsenic compounds, as well as to provide As⁵⁺ ion survival capacity to those Gram-negative bacteria that are not resistant to such ions, thus enabling them to perform bioremediation of arsenate ions.

That is, the cells of the untransformed wild Gram-negative bacteria lineages, which naturally exhibit moderate resistance to arsenic ions, perform the precipitation of arsenic within the cell and subsequent volatilization of toxic arsenic ions to the external medium. Recombinant cells derived from lineages which naturally exhibit moderate resistance to arsenic ions, besides containing such natural mechanism, also have acquired a second mechanism: the extracellular arsenic adsorption mechanism. As a result, these recombinant lineages show: 1) an increase in the resistance capacity to arsenic ions; 2) an increase in the capacity of binding with arsenic ions; 3) may be employed in arsenic bioremediation in a totally new way that excludes the release of toxic volatile arsenic ions; 4) the arsenic ions may be potentially desorbed.

In the next step, recombinant and wild type lineages were inoculated into sterile ultrapure water (Milli-Q) containing 1 mM of sodium arsenate (31.2 ppm of As⁵⁺) and incubated for different periods, in order to determine the minimum time required for considerable uptake of As⁵⁺ ions from the external environment. An enhancement in bioremediation of the solution was observed as a function of the incubation time, possibly due to the increased exposure of the ArsR protein to the arsenic ions.

The quantification of As⁵⁺ ions was directly performed in the microbial mass because the bioremediation ability refers to the amount of ions bound on the bacterial cell surface, rather than to the arsenic amount reduction measured in the solution. This is because noises inherent to the experiment, such as the metalloid binding on the tube walls, differences of pipetting and high volatility of the compound, may generate artifacts and inconsistent results in the experimental studies. Direct quantification in the microbial mass was carried out by atomic emission spectrometry by plasma inductively coupled (ICP-AES) at the end of different incubation periods. It was found that the C. metallidurans CH34/pCM-As cells cultured in sodium arsenate showed higher ability to bind As⁵⁺ ions when compared to the wild type cells. The same results were observed for the E. coli UT5600/pCM-As and E. coli UT5600 cells, where the recombinant cells showed significant higher ability in As⁵⁺ ions chelation when compared to the non-recombinant cells (without ArsR on the cellular surface).

The As⁵⁺ binding results showed that both E. coli UT5600 and C. metallidurans CH34 wild type cells were able to bind 18.5 mg of As⁵⁺ ions present in the water/g of bacterial dry mass. The recombinant C. metallidurans CH34/pCM-As cells showed a binding capacity of 1.114 g of As⁵⁺ ions/g of bacterial dry mass and the recombinant E. coli UT5600/pCM-As cells showed a binding capacity of 331.5 mg of As⁵⁺ ions/g of bacterial dry mass after 4 hours of incubation.

The E. coli UT5600/pCM-As and C. metallidurans CH34/pCM-As strains constructed in the present invention are excellent bioremediation agents for As⁵⁺ because, besides being highly resistant in colonizing environments containing this metalloid, they showed a significant ability to accumulate As⁵⁺ in the presence of water containing high concentrations of this ion. This fact opens up prospects of using the effluent itself containing the toxic agent as a culture medium for these bacteria, providing a concomitant bioremediation during cell growth.

The present invention was based on the expression and cell surface display of the ArsR protein in C. metallidurans CH34 by employing a recombinant molecular mechanism for the anchoring of ArsR, with a view to use the recombinant strain in the treatment of sites contaminated by arsenic.

The set of results, presented herein, enables us to affirm that the ArsR protein expression and anchoring on the surface of E. coli UT5600/pCM-As and C. metallidurans CH34/pCM-As is an appropriate strategy to optimize their capacity in binding As⁵⁺ and even the most toxic As³⁺ form, due to the ArsR highly specific affinity to bind to all the organic species as reported in the literature (ZHANG et al., 2009). The present invention also opens opportunities to use this broad spectrum system in other Gram-negative bacteria that have bioremediation potential, contributing to the development of new recombinant strains not yet reported.

The present application innovatively discloses the anchoring of the ArsR protein on the cellular surface of microorganisms, by investigating the binding potential of As⁵⁺ ions to the modified bacterial lineages. Therefore, this invention is indeed innovative for the construction of novel bacterial lineages containing the recombinant pCM-As plasmid of broad-spectrum for Gram-negative bacteria capable of expressing C. metallidurans CH34 ArsR protein on their cellular surface using the signal peptide and the anchorage domain of the Neisseria gonorrhoeae IgA protease secretion system, under the control of pan promoter from Bacillus subtilis.

In order to obtain the transgenic bacteria for the bioremediation of arsenic, the following steps were carried out.

Obtaining the C. metallidurans CH34 Chromosome 1 arsR Gene

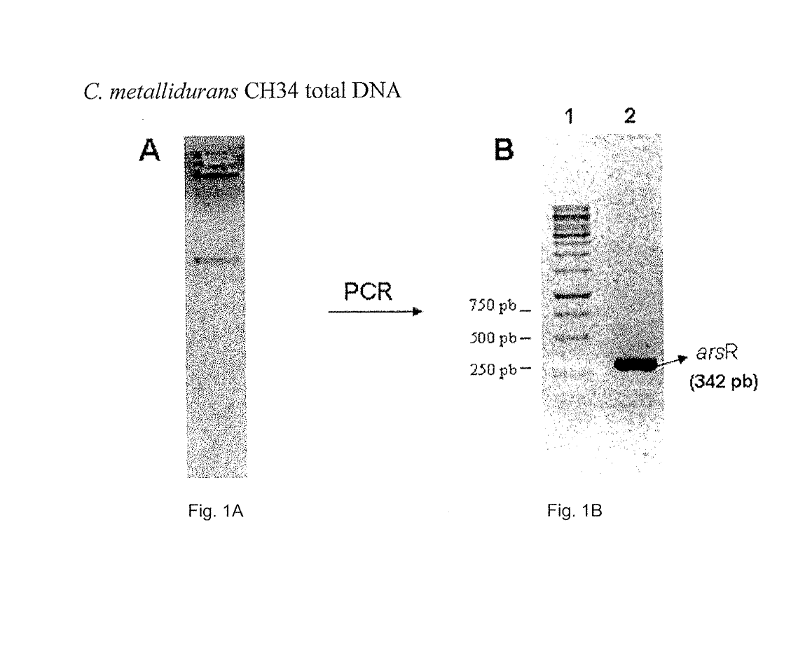

The total DNA of the C. metallidurans CH34 wild type strain was extracted according to TAGHAVI et al. (1994), visualized by electrophoresis on 0.8% agarose gel, and used as the DNA template to amplify the arsR gene (Gene ID 4037120) using the Polymerase Chain Reaction (PCR) (FIG. 1). To amplify the gene of interest from the total DNA of C. metallidurans CH34, a pair of primers was designed according to ZHANG et al (2009), comprising the sequences: 5′-TGCTCTAGAGCAATGGAAACCGAAAACGCTCT-3′ and 5′-ACGCGTCGACGGACTCCGTAGCGACTGAACA-3′ synthesized by Invitrogen, where the underlined nitrogenous bases correspond to the recognition sites for XbaI and SalI restriction enzymes, respectively. The primers above have as target the gene that encodes the regulatory ArsR protein of the ars operon of C. metallidurans CH34 present in chromosome 1, devoid of the tga stop codon. The PCR procedure was performed as described. The arsR gene (342 bp) was obtained without its stop codon and flanked by recognition sites for the XbaI and SalI enzymes (FIG. 1B).

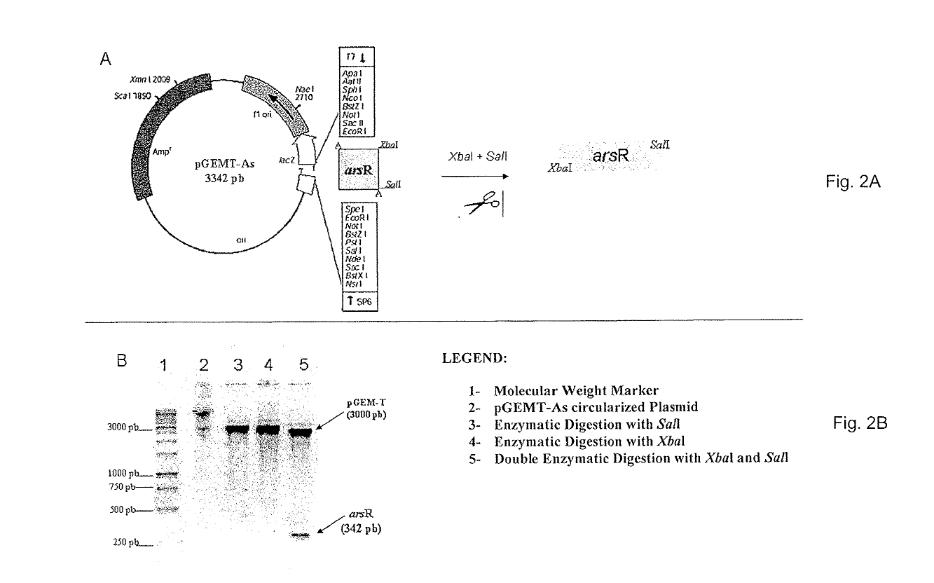

The arsR gene was inserted into the pGEM-T vector (3,000 bp) (Promega®) and the resulting plasmid, called PGEMT-As (3,342 bp) (FIG. 2) was employed for the genetic transformation (SAMBROOK; RUSSELL, 2001) of the E. coli DH5α strain (Promega®). The plasmid DNA of the transformants was isolated and subjected to double digestion with the XbaI and SalI enzymes to verify the presence of the arsR gene and confirm the construction (FIG. 2 B). Upon digestion, the PGEMT-As plasmid released a 342 bp fragment corresponding to the arsR gene endowed with XbaI and SalI cohesive ends. In the next step, this DNA fragment was purified and subcloned into the expression vector having the same cohesive ends.

FIGS. 2A and 2B illustrate the insertion of the arsR gene of C. metallidurans CH34 in the pGEM-T cloning vector.

FIG. 2A: Cloning of the arsR gene in the pGEM-T commercial vector (Promega®), yielding the pGEMT-As recombinant plasmid. After double digestion with XbaI and SalI, the gene was released with XbaI and SalI cohesive ends.

FIG. 2B: Colonies containing the pGEMT-As plasmid were chosen at random and had their plasmid DNAs analyzed by electrophoresis on 0.8% agarose gel. The plasmid preparations were analyzed employing enzymatic digestion with the pair of XbaI and SalI restriction enzymes, which confirmed the incorporation of the arsR insert in the pGEM-T plasmid (Lane 5). Lane 1 shows the migration profile of the molecular size marker (Gene O'ruler DNA 1 Kb—Fermentas®); Lane 2, the circularized pGEMT—As recombinant plasmid; Lane 3, the pGEMT-As plasmid digested only with the SalI enzyme, whereby the plasmid was linearized (3,342 bp); Lane 4, the pGEMT-As plasmid digested only with the XbaI enzyme, whereby the plasmid was linearized (3,342 bp); Lane 5, pGEMT-As double digested with XbaI and SalI enzymes, whereby the 342 bp arsR gene previously inserted was released. All these results provide evidences of the success of the construction.

Obtaining the Vector Containing the Heterologous Proteins Expression and Anchorage System for Gram-Negative Bacteria

The vector containing the heterologous proteins expression and anchoring system for Gram-negative bacteria derives from the pCM-Hg plasmid (PI1101557-8), which was originated from the pCM2 plasmid (PI0801282-2.) Since the pCM-Hg plasmid has in its sequence the gene of the C. metallidurans CH34 MerR protein, it was firstly necessary to remove this gene, which was flanked by recognition sites for the XbaI and SalI enzymes. Therefore, the pCM-Hg plasmid was digested with SalI and XbaI enzymes, which released the merR gene of 453 bp and resulted in a linear plasmid, named pCM with 6,490 bp, endowed with XbaI and SalI cohesive ends. The pCM plasmid carries the coding sequences of the signal peptide, the E-tag antigen, and of the β-domain of the N. gonorrhoeae IgA protease secretion system (FIG. 3).

The DNA fragment corresponding to the arsR gene, without the stop codon of protein synthesis, flanked by SalI and XbaI cohesive ends, previously isolated from the pGEMT-As plasmid, was inserted into the pCM expression vector that had been previously linearized with the same cohesive ends, to facilitate the ligation between insert and vector. This ligation mixture was used in the genetic transformation of the E. coli DH5α strain. The transformant clones were selected by growing them on solid medium LB+25 pg/mL chloramphenicol (Sigma-Aldrich). The migration profiles of plasmidial DNAs extracted from randomly selected clones were analyzed by agarose gel subjected to electrophoresis, allowing to select the bacterial colony where the desired recombinant plasmid was hosted. The newly constructed plasmid was named pCM-As (6,832 bp) (SEQ ID No 5). The DNA sequence corresponding to pan-promoter/signal peptide/arsR-/E-tag-/β-domain was denominated ARS-R anchorage cassette (2,233 bp), and the nucleotide sequence of this construct was analyzed by DNA sequencing (SEQ. ID No 3) (FIG. 3).

FIG. 3 is the representative scheme of the construction of the recombinant pCM-As plasmid. The arsR gene of C. metallidurans CH34 with SalI and XbaI cohesive ends, obtained by the pGEMT-As plasmid enzymatic digestion with XbaI/SalI enzymes, was inserted into the pCM expression vector (6,490 bp) (SEQ. ID No 4), using the T4 ligase enzyme (Fermentas®), giving rise to the pCM-As plasmid (6,832 bp) (SEQ. ID N° 5).

Expression Analysis of the arsR/e-Tag/B-Domain Fusion Protein (Under Pan promoter command) in E. coli UT5600 and C. metallidurans CH34

The ArsR anchorage cassette expression under the command of the pan promoter was evaluated in the E. coli UT5600/pCM-As and C. metallidurans CH34/pCM-As recombinant lineages The protein profile of each lineage was analyzed by SDS-PAGE 15%. Analysis of total protein profiles revealed that the recombinant lineages E. coli UT5600/PCM-As and C. metallidurans CH34/pCM-As showed an additional band of approximately 58 kDa, when compared to the correspondent wild type lineages, proving that the anchorage cassette was expressed in the recombinant lineages (FIG. 4A and FIG. 4 B, respectively).

FIGS. 4A and 4B show profiles of total proteins visualized by SDS-PAGE 15% stained with “Coomassie Blue R250.” A: 1—molecular weight marker (Prestained Protein Marker MW 20-120 kDa-Fermentas®), 2—E. coli UT5600, 3—E. coli UT5600/pCM-As. B: 1—molecular weight marker (Prestained Protein Marker MW 20-120 kDa—Fermentas®), 2—C. metallidurans CH34, 3—C. metallidurans CH34/pCM-As.

Functional Analysis of the Anchoring System in E. coli UT5600 and C. metallidurans CH34 Bacteria

The functional analysis of the anchoring system in E. coli UT5600/pCM-As and in C. metallidurans CH34/pCM-As was performed by fluorescence microscopy. For this assay, the primary anti-E-tag antibody produced in mice (GE Life Sciences) and the secondary FITC-conjugated anti-mouse antibody (Sigma-Aldrich) were used, for probing and for fluorescence emission, respectively. The obtained results showed that the E-tag antigen was transported to the external membrane of C. metallidurans CH34/pCM-As cells (FIG. 5B), and E. coli UT5600/pCM-As cells (FIG. 5D), by the appearance of fluorescent green signal emitted after the specific recognition reaction between antigen and antibody occurred. The correspondent non-recombinant lineages were used as negative controls of the experiment and showed no reactivity in the assay (FIGS. 5A and 5C, respectively).

FIGS. 5B and 5D show the results of the fluorescence microscopy assay where the E-tag antigen secretion was observed only in the recombinant strains C. metallidurans CH34/pCM-As and E. coli UT5600/pCM-As, respectively. A: C. metallidurans CH34; B: C. metallidurans CH34/pCM-As; C: E. coli UT5600; D: E. coli UT5600/pCM-As.

Analysis of arsR Protein Expression (Under Pan Promoter Command) and Anchorage on the External Membrane of E. coli UT5600

Proteins from E. coli UT5600/pCM-As recombinant cells were fractionated into Soluble Fraction (SF), Internal Membrane (IM) and External Membrane (EM). Wild type E. coli UT5600 was used as the negative control of the experiment. Cell fractionation was analyzed by 15% SDS-PAGE (FIG. 6A).

After electrophoresis, protein fractions were transferred from the polyacrylamide gel to a nitrocellulose membrane (Hybond C estra-Bio-Rad). A “Western blotting” assay was conducted using the primary anti-E-tag antibody produced in mice (-GE Life Sciences) and then, secondary IgG conjugated antibody with horseradish peroxidase, produced in mice (Sigma-Aldrich).

FIG. 6 shows the cell fractionation of E. coli UT5600 and E. coli UT5600/pCM-As, visualized by SDS-PAGE15% stained with “Coomassie Blue R250”. FIG. 6 B shows the “Western blotting” results of the various cell fractions after incubation with anti-E-tag antibody (primary commercial antibody produced in mice—GE Life Sciences) and secondary anti-mouse antibody, conjugated to horseradish peroxidase (secondary commercial antibody produced in mice and combined with horseradish peroxidase—Sigma-Aldrich).

FIG. 6A: SDS-PAGE 15% protein profiles of cell fractions of E. coli UT5600 and E. coli UT5600/pCM-As: 1—molecular size marker (Prestained Protein Marker 20-120 kDa MW-Fermentas), 2—Soluble Fraction (SF) of E. coli UT5600; 3—Soluble Fraction (SF) of E. coli UT5600/pCM-As; 4—Internal Membrane Fraction (IM) of E. coli UT5600; 5—Internal Membrane Fraction (IM) of E. coli UT5600/pCM-As; 6—External Membrane Fraction (EM) of E. coli UT5600; 7—External Membrane Fraction (EM) of E. coli UT5600/pCM-As; 8—molecular size marker (Page-Ruler Unstained Protein Marker 10-200 kDa, Fermentas®). The electrophoretic analysis showed an additional band of approximately 58 kDa, corresponding to the protein fusion β-domain of the IgA protease secretion system (45.2 kDa) (VEIGA et al., 2002), E-tag epitope (1.4 kDa), and ArsR protein (11.4 kDa) in the proteins of the external membrane fraction of the recombinant strain (lane 7). The 58 kDa band was not seen in the external membrane fraction of the untransformed strain (lane 6).

FIG. 6 B: “Western Blotting” Assay: 1—molecular size marker (Prestained Protein Marker 20-120 kDa MW—Fermentas®)—(SF) E. coli UT5600; 3—(SF) E. coli UT5600/pCM-As; 4—(IM) E. coli UT5600; 5—(IM) E. coli UT5600/pCM-As; 6—(EM) E. coli UT5600; 7—(EM) E. coli UT5600/pCM-As; 8—molecular size marker (Page-Ruler Unstained Protein Marker 10-200 kDa—Fermentas®). Reactivity was observed only in the external membrane fraction of the recombinant E. coli UT5600/pCM-As cells, (lane 7), confirming the expression of the fusion protein ArsR/E-tag/β-domain in the external membrane of recombinant bacteria.

Analysis of arsR Protein Expression (Under Pan Promoter Command) and Anchorage on the External Membrane of C. metallidurans CH34

To evaluate the expression and location of the ArsR protein in the external membrane of the C. metallidurans CH34/PCM-As recombinant lineage, the total protein extract was fractionated in: Soluble Fraction (SF), Inner Membrane (IM), and External Membrane (EM). Cell fractionation of total protein extract of wild type cells was used as the negative control of the experiment. The different cell fractions obtained for the recombinant and wild type cells were visualized by SDS-PAGE (FIG. 7A). After electrophoretic analysis, proteins from the different fractions were transferred from the polyacrylamide gel to a nitrocellulose membrane and the expression of the fusion protein ArsR/Etag/β-domain in the external membrane of the recombinant cells was confirmed by “Western Blotting” using the E-tag epitope as a reporter, which is recognized with specificity by the primary antibody anti-E-tag produced in mouse (GE Life Sciences) and anti-mouse secondary antibody, conjugated with horseradish peroxidase (Sigma-Aldrich) (FIG. 7B). The results demonstrated that the E-tag was detected only in the external membrane fraction of C. metallidurans CH34/PCM-As cells, indicating that indeed the protein is bound to the bacterium external membrane. (FIG. 7B).

FIG. 7A: SDS-PAGE 15% protein profiles of cell fractions of C. metallidurans CH34 and C. metallidurans CH34/pCM-As. 1—molecular size marker (Prestained Protein MW Marker 20-120 kDa—Fermentas®); 2—(SF) C. metallidurans CH34; 3—(SF) C. metallidurans CH34/pCM-As; 4—(IM) C. metallidurans CH34; 5—(IM) C. metallidurans CH34/pCM-As; 6—(EM) C. metallidurans CH34; 7—(EM) C. metallidurans CH34/pCM-As; 8—molecular size marker (Page-Ruler Unstained Protein Marker 10-200 kDa—Fermentas®). The electrophoretic analysis showed an additional band of approximately 58 kDa, corresponding to the fusion protein β-domain of the IgA protease secretion system (45.2 kDa) (VEIGA et al., 2002), E-tag epitope (1.4 kDa), and ArsR protein (11.4 kDa) in the proteins of the external membrane fraction of the recombinant strain (lane 7). The 58 kDa band was not seen in the external membrane fraction of the untransformed strain (lane 6).

FIG. 7B: “Western-blotting” Assay: 1—molecular size marker (Prestained Protein Marker 20-120 kDa MW—Fermentas®); 2—(SF) C. metallidurans CH34; 3—(SF) C. metallidurans CH34/pCM-As; 4—(IM) C. metallidurans CH34; 5—(IM) C. metallidurans CH34/pCM-As; 6—(EM) C. metallidurans CH34; 7—(EM) C. metallidurans CH34/pCM-As; 8—molecular size marker (Page-Ruler Unstained Protein Marker 10-200 kDa—Fermentas®). Reactivity was observed only in the external membrane fraction of the recombinant C. metallidurans CH34/pCM-As cells, (lane 7), confirming the expression of the protein fusion ArsR/E-tag/β-domain in the external membrane of the recombinant bacteria. In fact, the 58 kDa band, corresponding to the positive reaction of antigen (E-tag)-antibody interaction was visualized only in lane 7.

Analysis of the Binding Capacity of AS5+ Ions by the Recombinant C. metallidurans/PCM-as Cells in the Presence of 500 Mm Sodium Arsenate.

To analyze their capability to adsorb arsenate ions, C. metallidurans CH34/PCM-As cells were incubated in 500 mM sodium arsenate for 2 hours and visualized by Transmission Electron Microscopy (TEM). The recombinant cells showed the presence of aggregates bound to the external membrane, indicating a significant bioaccumulation of arsenate ions on the cellular surface, demonstrating that, in fact, the presence of the ArsR protein increased the cells capability to bind As⁵⁺ ions (FIG. 8D).

FIG. 8 shows the images obtained by TEM (X 40K) of bacterial cells: 8A—C. metallidurans CH34 after incubation in (Milli-Q) ultrapure water; 8 B—C. metallidurans CH34 after incubation in 500 mM sodium arsenate, 8 C—C. metallidurans CH34/pCM-As after incubation in (Milli-Q) ultrapure water—8 D—C. metallidurans CH34/pCM-As after incubation in 500 mM sodium arsenate. In FIGS. 8C and 8D, the intracellular precipitationof As⁵⁺ ions was observed. FIG. 8D also shows a strong accumulation of As⁵⁺ ions on the cellular surface of the recombinant cells, compared to that observed in C. metallidurans CH34 untransformed cells (FIG. 8B).

Analysis of the Binding Capacity of AS5+ Ions by the Recombinant E. coli UT5600/PCM-As Cells in the Presence of 500 mm Sodium Arsenate.

To analyze their adsorption ability of arsenate ions, E. coli UT5600/PCM-As cells were incubated in 500 mM sodium arsenate for 2 hours and visualized by Transmission Electron Microscopy (TEM). The recombinant cells showed the presence of aggregates bound to the external membrane, indicating a significant bioaccumulation of arsenate ions on the cellular surface, demonstrating that, in fact, the presence of the ArsR protein increased the cells capability to bind As⁵⁺ ions. (FIG. 9D).

FIG. 9 shows the images obtained by TEM (40,000× magnification) of bacterial cells: 9A—E. coli UT5600 after incubation in (Milli-Q) ultrapure water, 9B—E. coli UT5600 after incubation in 500 mM sodium arsenate, where intracellular precipitation of As⁵⁺ ions can be observed; 9C—E. coli UT5600/pCM-As after incubation in (Milli-Q) ultrapure water; 9D—E coli UT5600/pCM-As after incubation in 500 mM sodium arsenate, where intracellular precipitation of As⁵⁺ ions and a large increase in accumulation of As⁵⁺ on the cellular surface can be observed.

Analysis of the Increase in Arsenate Resistance Promoted by the Insertion of the PCM-As Plasmid in the E. coli UT5600 Lineage

To find out whether the recombinant E. coli UT5600/pCM-As lineage had increased resistance to arsenate ions, as compared to the UT5600 lineage from which it is derived, the MIC against Na₃As0₄ of each of the lineages was determined.

The MIC of the E. coli UT5600 cells was 25 mM Na₃As0₄, indicating that this lineage has a high natural resistance to As⁵⁺ ions (FIG. 10A). The recombinant E. coli UT5600/pCM-As lineage showed a MIC of 50 mM Na₃As0₄, representing a survivability 100% higher than that of the wild lineage (FIG. 10B). The final bacterial growth in different Na₃As0₄ concentrations was quantified by Absorbance reading at 600 ηm (FIG. 10C). The assays were performed in triplicate, showing similar results.

Analysis of the Increase in Arsenate Resistance Promoted by the Insertion of the pCM-As Plasmid in the C. metallidurans CH34 Lineage

The MIC of C. metallidurans CH34 and C. metallidurans CH34/pCM-As cells against As5+ ions were also studied. The MIC of Na₃As04 for C. metallidurans CH34 was 500 mM, indicating that the wild type lineage has a high natural resistance to arsenate (FIG. 11A). The MIC of Na₃As04 for C. metallidurans CH34/pCM-As was above 1,000 mM, indicating an increase in survivability to As5+ ions above 100% (FIG. 11B). The bacterial growth for the MIC assays was quantified by Absorbance reading at 600 nm (FIG. 11C). The assays were performed in triplicate, showing similar results.

Evaluation of C. metallidurans CH34/PCM-As Cells Ability to Adsorb AS5+ Ions

The evaluation of the As⁵⁺ ions adsorption capability by the C. metallidurans CH34/pCM-As cells was performed by incubating 0.02 g of bacterial dry weight in 10 mL of 1 mM sodium arsenate for different times (0, 10, 30, 60, 120, and 240 minutes), under stirring at room temperature. After each incubation period, the quantification of arsenate in the microbial mass was performed by inductively coupled plasma atomic emission spectrometry (ICP-AES). The results showed that the biosorption of pentavalent arsenic by C. metallidurans CH34, was 18,500 μg of As⁵⁺/g dry weight (i.e. 0.018 g As⁵⁺/g dry weight) after 240 min of incubation. The recombinant C. metallidurans CH34/pCM-As cells were able to bind 1,114,000 μg As⁵⁺/g dry weight (i.e. As⁵⁺ 1.11 g/g dry weight) in the same period, indicating that the recombinant bacterium carrying the pCM-As plasmid has 60 times higher capacity to bind As⁵⁺ than the control lineage (FIG. 12).

Evaluation of E. coli UT5600/PCM-As Cells Ability to Adsorb AS5+ Ions

The evaluation of As⁵⁺ ions adsorption capacity by E. coli UT5600/pCM-As cells was carried out following the same procedure used for C. metallidurans CH34/pCM-As cells. 0.02 g of E. coli UT5600/pCM-As dry mass were incubated in 10 mL of 1 mM sodium arsenate. Incubation was carried out at different times (0, 10, 30, 60, 120, and 240 minutes), under stirring, at room temperature. After each incubation period, the quantification of arsenate in the microbial mass was performed by inductively coupled plasma atomic emission spectrometry (ICP-AES). It was found that the As⁵⁺ adsorption by E. coli UT5600 was 18,500 pg of As⁵⁺/g dry weight (i.e. 0.018 g As⁵⁺/g dry weight) in 240 minutes. E. coli UT5600/pCM-As cells were able to bind 331,500 μg of As⁵⁺/g dry weight (i.e. 0.33 g of As⁵⁺/g dry weight) in the same period, showing 18 times higher ability to accumulate arsenate ions than the control lineage (FIG. 13). In short, E. coli UT5600/pCM-As was able to accumulate about 18 times more pentavalent arsenic than the wild type E. coli UT5600 lineage, simply due to the fact that it contains the pCM-As plasmid constructed according to the present invention.

All recombinant lineages constructed in the present invention showed better performance after 240 min of incubation in a solution containing As⁵⁺ ions, in the conditions in which the assays were performed. However, this incubation time could be decreased by optimizing the assay conditions. It was also verified that the cell viability after the experiment, in all cases was of 100%.

Comparison Between C. metallidurans CH34/PCM-As and E. coli UT5600/PCM-As Lineages Capability to Adsorb AS5+ Ions

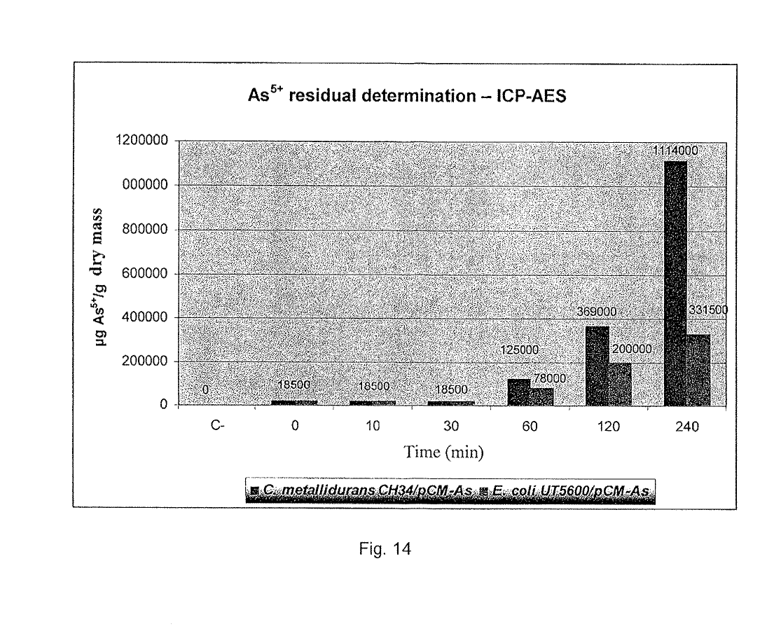

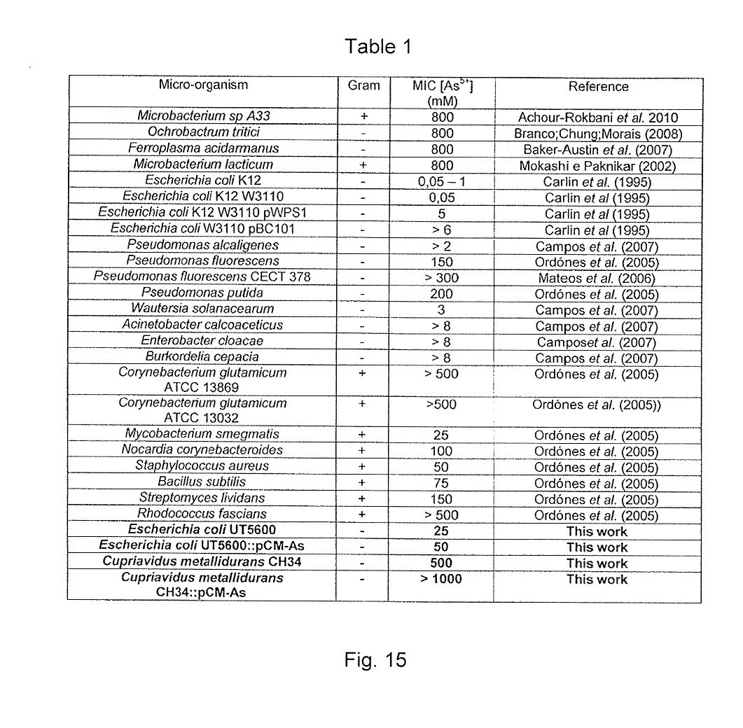

The comparison of the As⁵⁺ ions adsorption ability of E. coli UT5600/pCM-As and C. metallidurans CH34/pCM-As bacteria shows that, after 240 minutes, C. metallidurans CH34/pCM-As has three times greater ability of biosorption than E. coli UT5600/pCM-As. In fact, the C. metallidurans CH34/pCM-As cells were found to be always more effective in binding arsenate ions (FIG. 14).

As shown in Table 1, the bacterial strain C. metallidurans CH34/pCM-As can be considered the most arsenate-resistant bacterium ever reported.

1. A GENE comprising an arsR gene without the stop codon of protein synthesis (of SEQ. ID No 1).

2. The GENE, according to claim 1, wherein the gene encodes a protein of high affinity and specificity to arsenic ions.

3. An ARS-R ANCHORAGE CASSETTE, comprising SEQ. ID No 3.

4. The ARS-R ANCHORAGE CASSETTE, according to claim 3, further comprising a signal peptide encoding sequence, an ArsR protein encoding sequence, an E-tag encoding sequence and the Neisseria gonorrhoeae IgA protease β-domain encoding sequence.

5. The ARS-R ANCHORAGE CASSETTE, according to claim 4, wherein the arsR anchorage cassette expresses the fusion sequence under translational control of the pan promoter.

6. A RECOMBINANT PLASMID, according to claim 1, comprising SEQ. ID No 5.

7. The RECOMBINANT PLASMID, according to claim 6, comprising the encoding gene sequence of arsR expression-anchorage cassette of SEQ. ID No 4.

8. The RECOMBINANT PLASMID, according to claim 6, comprising the gene sequence which encodes an anchorage system of an arsenic chelant protein in the cellular surface of Gram-Negative bacteria.

9. The RECOMBINANT PLASMID, according to claim 6, providing arsenic resistance in Gram-negative bacteria sensitive to the metalloid.

10. A BACTERIAL TRANSGENIC LINEAGE, according to claim 8, wherein the bacteria comprises, preferably, Escherichia coli and Cupriavidus metallidurans.

11. A BACTERIAL TRANSGENIC LINEAGE, according to claim 1, replicating the recombinant plasmid and expresses the arsR anchorage cassette in high basal levels.

12. USE OF THE GENE, according to claim 1, encoding a protein capable of binding to metal and metalloid ions.

13. USE OF THE GENE according to claim 11, wherein the metalloid ions comprise, specifically, As⁵⁺ or As³⁺,

14. USE OF THE BACTERIAL LINEAGE, according to claim 9, employed in environmental bioremediation processes of arsenic compounds.

15. The gene sequence according to claim 1, comprising the arsR gene of SEQ. ID No 2 without the stop codon of protein synthesis, inserted into a cloning vector.

16. The gene sequence, according to claim 1, wherein the gene encodes a protein of high affinity and specificity to metal and metalloid ions.

17. The gene sequence of claim 16, wherein the metalloid ions comprise at least one of arsenate and arsenite ions.

18. A recombinant plasmid, containing the gene sequence defined by claim 1.

19. A recombinant plasmid, comprising an encoding gene sequence which allows the expression and cell surface display of any desired protein in bacteria (SEQ. ID No 4).

20. The recombinant plasmid according to claim 1, wherein the recombinant plasmid confers to a host bacteria enhanced arsenic resistance and enhanced capability to adsorb arsenic ions (SEQ. ID No 5).

21. The recombinant plasmid according to claim 20, containing the gene sequence which encodes an anchorage system of an arsenic chelating protein in the cellular surface of Gram-negative bacteria.

22. The recombinant plasmid according to claim 19, wherein the recombinant plasmid provides enhanced arsenic resistance and enhanced capability to adsorb arsenic ions in Gram-negative bacteria.

23. A bacterial transgenic lineage comprising the recombinant plasmid defined in claim 20.

24. The bacterial transgenic lineage according to claim 23, wherein the exemplified lineage comprises Escherichia coli or Cupriavidus metaffidurans.

25. The bacterial transgenic lineage according to claim 23, wherein the lineage expresses the ARS-R anchorage cassette, either under inducing or under non-inducing culture conditions.

26. Use of the recombinant plasmid, according to claim 20, wherein the recombinant plasmid confers enhanced arsenic resistance and enhanced capability to adsorb arsenic ions in Gram-negative bacteria.


SEQUENCE LISTING (5 sequences)

SEQ ID NO: 1 - DNA, 342 bp, Artificial Sequence
  Description of Artificial Sequence Synthetic polynucleotide
tctagagcaatggaaaccgaaaacgctcttgaagccctcgccgcgctggcccacggcatccgcctggccgttttccgcctgcttgtgcaggccggccccgaagggctgccggctggccgtattgccgaactgatggagatgccagcgtcgtcgctatcgttccacctcaaggaactgcaccgcgccggactgctggcgagccgtcaggaaggccgctcgatcatctacatggctcaattcgaaaccatgaacgccttgctgggctatctcacggaaaactgttgtggcggcgcgccgtgttcaccggtgtcctcctgttcagtcgctacggagtccgtcgac

SEQ ID NO: 2 - DNA, 3342 bp, Artificial Sequence
  Description of Artificial Sequence Synthetic polynucleotide
gggcgaattgggcccgacgtcgcatgctcccggccgccatggccgcgggattctagagcaatggaaaccgaaaacgctcttgaagccctcgccgcgctggcccacggcatccgcctggccgttttccgcctgcttgtgcaggccggccccgaagggctgccggctggccgtattgccgaactgatggagatgccagcgtcgtcgctatcgttccacctcaaggaactgcaccgcgccggactgctggcgagccgtcaggaaggccgctcgatcatctacatggctcaattcgaaaccatgaacgccttgctgggctatctcacggaaaactgttgtggcggcgcgccgtgttcaccggtgtcctcctgttcagtcgctacggagtccgtcgacatcactagtgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 3 - DNA, 2233 bp, Bacillus subtilis
gagctccaccgcggttcggtatcgaaagccgctttgatcgagccgggctcatacaagacatcgatctggtcaacgtcattatcaaattttatgtcggtgcctcgtgcagattcagcctatcatcaatactataaaaagatcctttcctatgttcaaaaaaacggagaagaaatcggagatccccaagaggttgccgacctcatttatcaattggcaacaaaacacgacataaagaatttgcgatacccgatcggaaagggcatcaagctcaccctgctgttccgatcgctttttccttggtctgcgtgggagtctatcctgaagaaaaagctattcagctgatctaaattataattattataatttagtattgatttttatttagtatatatataattaagtcaacagatcacaaggaggacgttatcatatgaaatacctattgcctacggcagccgctggattgttattactcgcggcctgctctagatctagagcaatggaaaccgaaaacgctcttgaagccctcgccgcgctggcccacggcatccgcctggccgttttccgcctgcttgtgcaggccggccccgaagggctgccggctggccgtattgccgaactgatggagatgccagcgtcgtcgctatcgttccacctcaaggaactgcaccgcgccggactgctggcgagccgtcaggaaggccgctcgatcatctacatggctcaattcgaaaccatgaacgccttgctgggctatctcacggaaaactgttgtggcggcgcgccgtgttcaccggtgtcctcctgttcagtcgctacggagtccgtcgacgtcgacggtgcgccggtgccgtatccggatccgctggaaccgatcgacaattcagccgcaattagtatggcaaatccacgtccaccaacaccgcgggctgctgcggccgtattttcattggatgattatgatgcaaaagacaatagtgaatcatcaataggtaatttagctcgtgtaatacctagaatgggaagggagttaattaatgattatgaagaaatccccttggaggagttggaagatgaagcggaagaagaacgtcgccaagcaacgcaattccactccaaaagtcgtaaccgtagagctatatcatcggaaccatcatctgatgaagatgcatctgaatcggtttccacatcagacaaacaccctcaagataatacggaacttcatgaaaaagttgagacggcgggtttacaaccaagagccgcgcagccgcgaacccaagccgccgcgcaagccgatgcagtcagcaccaatactaactcggctttatctgacgcaatggcaagcacgcaatctatcttgttggatacaggtgcttacttaacacggcacattgcacaaaaatcacgcgctgatgccgaaaaaaacagtgtttggatgtcaaacaccggttatggccgtgattatgcttccgcacaatatcgccggtttagttcgaaacgcacgcaaacacaaatcggcattgaccgcagcttgtccgaaaatatgcagataggcggagtattgacttactctgacagtcagcatacttttgatcaggcgggcggcaaaaatacttttgtgcaacggaacctttatggtaagtattatttaaatgatgcttggtatgtggccggcgatattggtgcgggcagcttgagaagccggttacaaacgcagcaaaaagcaaactttaaccgaacaagcatccaaaccggccttactttgggcaatacgctgaaaatcaatcaattcgagattgtccctagtgcgggtatccgttacagccgcctgtcatctgcagattacaagttgggtgacgacagtgttaaagtaagttctatggcagtgaaaacactaacggccggactggattttgcttatcggtttaaagtcggcaaccttaccgtaaaacccttgttatctgcagcttactttgccaattatggcaaaggcggcgtgaatgtgggcggtaaatccttcgcctataaagcagataatcaacagcaatattcagcaggcgtcgcgttactgtaccgtaatgttacattaaacgtaaatggcagtattacaaaaggaaaacaattggaaaaacaaaaatccggacaaattaaaatacagattcgtttctaaaagcttggaagggcgaattccagcacactggcggccgttactagtggatccgagctcggtacc

SEQ ID NO: 4 - DNA, 6490 bp, Artificial Sequence
  Description of Artificial Sequence Synthetic polynucleotide
gtcgacggtgcgccggtgccgtatccggatccgctggaaccgatcgacaattcagccgcaattagtatggcaaatccacgtccaccaacaccgcgggctgctgcggccgtattttcattggatgattatgatgcaaaagacaatagtgaatcatcaataggtaatttagctcgtgtaatacctagaatgggaagggagttaattaatgattatgaagaaatccccttggaggagttggaagatgaagcggaagaagaacgtcgccaagcaacgcaattccactccaaaagtcgtaaccgtagagctatatcatcggaaccatcatctgatgaagatgcatctgaatcggtttccacatcagacaaacaccctcaagataatacggaacttcatgaaaaagttgagacggcgggtttacaaccaagagccgcgcagccgcgaacccaagccgccgcgcaagccgatgcagtcagcaccaatactaactcggctttatctgacgcaatggcaagcacgcaatctatcttgttggatacaggtgcttacttaacacggcacattgcacaaaaatcacgcgctgatgccgaaaaaaacagtgtttggatgtcaaacaccggttatggccgtgattatgcttccgcacaatatcgccggtttagttcgaaacgcacgcaaacacaaatcggcattgaccgcagcttgtccgaaaatatgcagataggcggagtattgacttactctgacagtcagcatacttttgatcaggcgggcggcaaaaatacttttgtgcaacggaacctttatggtaagtattatttaaatgatgcttggtatgtggccggcgatattggtgcgggcagcttgagaagccggttacaaacgcagcaaaaagcaaactttaaccgaacaagcatccaaaccggccttactttgggcaatacgctgaaaatcaatcaattcgagattgtccctagtgcgggtatccgttacagccgcctgtcatctgcagattacaagttgggtgacgacagtgttaaagtaagttctatggcagtgaaaacactaacggccggactggattttgcttatcggtttaaagtcggcaaccttaccgtaaaacccttgttatctgcagcttactttgccaattatggcaaaggcggcgtgaatgtgggcggtaaatccttcgcctataaagcagataatcaacagcaatattcagcaggcgtcgcgttactgtaccgtaatgttacattaaacgtaaatggcagtattacaaaaggaaaacaattggaaaaacaaaaatccggacaaattaaaatacagattcgtttctaaaagcttggaagggcgaattccagcacactggcggccgttactagtggatccgagctcggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgaagacgaaagggcctcgtgatacgcctatttttataggttaatgtcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggttcggtatcgaaagccgctttgatcgagccgggctcatacaagacatcgatctggtcaacgtcattatcaaattttatgtcggtgcctcgtgcagattcagcctatcatcaatactataaaaagatcctttcctatgttcaaaaaaacggagaagaaatcggagatccccaagaggttgccgacctcatttatcaattggcaacaaaacacgacataaagaatttgcgatacccgatcggaaagggcatcaagctcaccctgctgttccgatcgctttttccttggtctgcgtgggagtctatcctgaagaaaaagctattcagctgatctaaattataattattataatttagtattgatttttatttagtatatatataattaagtcaacagatcacaaggaggacgttatcatatgaaatacctattgcctacggcagccgctggattgttattactcgcggcctgctctaga

SEQ ID NO: 5 - DNA, 6832 bp, Artificial Sequence
  Description of Artificial Sequence Synthetic polynucleotide
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggttcggtatcgaaagccgctttgatcgagccgggctcatacaagacatcgatctggtcaacgtcattatcaaattttatgtcggtgcctcgtgcagattcagcctatcatcaatactataaaaagatcctttcctatgttcaaaaaaacggagaagaaatcggagatccccaagaggttgccgacctcatttatcaattggcaacaaaacacgacataaagaatttgcgatacccgatcggaaagggcatcaagctcaccctgctgttccgatcgctttttccttggtctgcgtgggagtctatcctgaagaaaaagctattcagctgatctaaattataattattataatttagtattgatttttatttagtatatatataattaagtcaacagatcacaaggaggacgttatcatatgaaatacctattgcctacggcagccgctggattgttattactcgcggcctgctctagatctagagcaatggaaaccgaaaacgctcttgaagccctcgccgcgctggcccacggcatccgcctggccgttttccgcctgcttgtgcaggccggccccgaagggctgccggctggccgtattgccgaactgatggagatgccagcgtcgtcgctatcgttccacctcaaggaactgcaccgcgccggactgctggcgagccgtcaggaaggccgctcgatcatctacatggctcaattcgaaaccatgaacgccttgctgggctatctcacggaaaactgttgtggcggcgcgccgtgttcaccggtgtcctcctgttcagtcgctacggagtccgtcgacgtcgacggtgcgccggtgccgtatccggatccgctggaaccgatcgacaattcagccgcaattagtatggcaaatccacgtccaccaacaccgcgggctgctgcggccgtattttcattggatgattatgatgcaaaagacaatagtgaatcatcaataggtaatttagctcgtgtaatacctagaatgggaagggagttaattaatgattatgaagaaatccccttggaggagttggaagatgaagcggaagaagaacgtcgccaagcaacgcaattccactccaaaagtcgtaaccgtagagctatatcatcggaaccatcatctgatgaagatgcatctgaatcggtttccacatcagacaaacaccctcaagataatacggaacttcatgaaaaagttgagacggcgggtttacaaccaagagccgcgcagccgcgaacccaagccgccgcgcaagccgatgcagtcagcaccaatactaactcggctttatctgacgcaatggcaagcacgcaatctatcttgttggatacaggtgcttacttaacacggcacattgcacaaaaatcacgcgctgatgccgaaaaaaacagtgtttggatgtcaaacaccggttatggccgtgattatgcttccgcacaatatcgccggtttagttcgaaacgcacgcaaacacaaatcggcattgaccgcagcttgtccgaaaatatgcagataggcggagtattgacttactctgacagtcagcatacttttgatcaggcgggcggcaaaaatacttttgtgcaacggaacctttatggtaagtattatttaaatgatgcttggtatgtggccggcgatattggtgcgggcagcttgagaagccggttacaaacgcagcaaaaagcaaactttaaccgaacaagcatccaaaccggccttactttgggcaatacgctgaaaatcaatcaattcgagattgtccctagtgcgggtatccgttacagccgcctgtcatctgcagattacaagttgggtgacgacagtgttaaagtaagttctatggcagtgaaaacactaacggccggactggattttgcttatcggtttaaagtcggcaaccttaccgtaaaacccttgttatctgcagcttactttgccaattatggcaaaggcggcgtgaatgtgggcggtaaatccttcgcctataaagcagataatcaacagcaatattcagcaggcgtcgcgttactgtaccgtaatgttacattaaacgtaaatggcagtattacaaaaggaaaacaattggaaaaacaaaaatccggacaaattaaaatacagattcgtttctaaaagcttggaagggcgaattccagcacactggcggccgttactagtggatccgagctcggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgaagacgaaagggcctcgtgatacgcctatttttataggttaatgtcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt